SEQUENCE LISTING (14 sequences)

SEQ ID NO: 1 - protein, 528 aa, synthetic construct
  REGION 1..528: note = synthetic polymer
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 2 - protein, 533 aa, synthetic construct
  REGION 1..533: note = synthetic polymer
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDAAHLAASPPPLALLAGAMLLLLAPTLY

SEQ ID NO: 3 - protein, 503 aa, synthetic construct
  REGION 1..503: note = synthetic polymer
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPAGTTD

SEQ ID NO: 4 - protein, 506 aa, synthetic construct
  REGION 1..506: note = synthetic polymer
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 5 - protein, 754 aa, synthetic construct
  REGION 1..754: note = synthetic polymer
MQGACVLLLLGLHLQLSLGLIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDGGSGGSGGGGSGGGGSEPKSCDKTHTCPPCPAPELLGGPSVFLFPPKPKDTLMISRTPEVTCVVVDVSHEDPQVKFNWYVDGVQVHNAKTKPREQQYNSTYRVVSVLTVLHQNWLDGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 534 aa, synthetic construct
  REGION 1..534: note = synthetic polymer
MQWACVLLLLGLWLQLSLTFIPAEEEDPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYKTIGVSAAARYNQCNTTSGNEVTSVMNRAKKAGKSVGVVTTSRVQHASPAGAYAHTVNRNWYSDADLPADAQTYGCQDIATQLVNNMDIDVILGGGRMYMFPEGTPDPEYPYDVNQTGVRKDKRNLVQEWQAKHQGAQYVWNRTELLQAANDPSVTHLMGLFEPADMKYNVQQDPTKDPTLEEMTEAALQVLSRNPQGFYLFVEGGRIDHGHHEGKAYMALTDTVMFDNAIAKANELTSELDTLILATADHSHVFSFGGYTLRGTSIFGLAPSKASDNKSYTSILYGNGPGYVLGGGLRPDVNDSISEDPSYRQQAAVPLSSESHGGEDVAVFARGPQAHLVHGVQEETFVAHVMAFAGCVEPYTDCNLAPPAGTTDAAHPIEGRSVVPALLPLRAGTLLLLETATAP

SEQ ID NO: 7 - DNA, 1669 bp, synthetic construct
  misc_feature 1..1669: note = synthetic polymer
  source 1..1669: mol_type = other DNA
atgcaggggccctgggtgctgctgctgctgggcctgaggctacagctctccctgggcgtcatcccaggtaatgaggctccccaagctgttccacacacagggcaccccctcagccaggctgacctgatctctactctccccctggccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctga

SEQ ID NO: 8 - DNA, 1671 bp, synthetic construct
  misc_feature 1..1671: note = synthetic polymer
  source 1..1671: mol_type = other DNA
atgcaggggccctgggtgctgctgctgctgggcctgaggctacagctctccctgggcgtcatcccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatccaccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcactcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgcctcccgcctgcaccaccgacgccgcgcacccagttgccgcgtcgctgccactgctggccgggaccctgctgctgctgggggcgtccgctgctccctgatttactaaaaccttgaaataaaattgtaaaacatcagtttgaaggcctgactctcagggtagttcttttttaattctgggtttt

SEQ ID NO: 9 - DNA, 1259 bp, synthetic construct
  misc_feature 1..1259: note = synthetic polymer
  source 1..1259: mol_type = other DNA
aaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgaacaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcaggaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaa

SEQ ID NO: 10 - protein, 420 aa, synthetic construct
  REGION 1..420: note = synthetic polymer
KKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKNRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

SEQ ID NO: 11 - protein, 487 aa, synthetic construct
  REGION 1..487: note = synthetic polymer
LIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPD

SEQ ID NO: 12 - protein, 506 aa, synthetic construct; identical to SEQ ID NO: 4
  REGION 1..506: note = synthetic polymer

SEQ ID NO: 13 - protein, 514 aa, synthetic construct
  REGION 1..514: note = synthetic polymer
LIPAEEENPAFWNRQAAQALDVAKKLQPIQTAAKNVILFLGDGMGVPTVTATRILKGQMNGKLGPETPLAMDQFPYVALSKTYNVDRQVPDSAGTATAYLCGVKGNYRTIGVSAAARYNQCNTTRGNEVTSVINRAKKAGKAVGVVTTTRVQHASPAGAYAHTVNRNWYSDADLPADAQKNGCQDIAAQLVYNMDIDVILGGGRMYMFPEGTPDPEYPDDASVNGVRKDKQNLVQEWQAKHQGAQYVWNRTALLQAADDSSVTHLMGLFEPADMKYNVQQDHTKDPTLAEMTEAALQVLSRNPRGFYLFVEGGRIDHGHHDGKAYMALTEAIMFDNAIAKANELTSELDTLILVTADHSHVFSFGGYTLRGTSIFGLAPGKALDSKSYTSILYGNGPGYALGGGSRPDVNGSTSEEPSYRQQAAVPLASETHGGEDVAVFARGPQAHLVHGVQEETFVAHIMAFAGCVEPYTDCNLPAPATATSIPDAAHLAASPPPLALLAGAMLLLLAPTLY

SEQ ID NO: 14 - protein, 533 aa, synthetic construct; identical to SEQ ID NO: 2
  REGION 1..533: note = synthetic polymer

(19) United States
(12) Patent Application Publication
(10) Pub. No.: US 2026/0048103 A1
(43) Pub. Date: Feb. 19, 2026

(54) TREATMENT OR PREVENTION OF GASTROINTESTINAL IMMUNOTHERAPY SIDE EFFECTS

(71) Applicant: Theriva Biologics, Inc., Rockville, MD (US)

(72) Inventors: Michael KALEKO, Rockville, MD (US); Christian FURLAN FREGUIA, Rockville, MD (US)

(21) Appl. No.: 19/101,977

(22) PCT Filed: Aug. 8, 2023
(86) PCT No.: PCT/US2023/071834

§ 371 Date: Feb. 7, 2025

Related U.S. Application Data
(60) Provisional application No. 63/396,398, filed on Aug. 9, 2022.

(51) Int. Cl. A61K 38/46; A61K 45/06

(52) U.S. Cl. CPC A61K 38/465; A61K 45/06; C12Y 301/03001

(57) ABSTRACT

The present disclosure relates, inter alia, to methods for the prevention and/or reduction of immune checkpoint inhibitor-mediated gastrointestinal side effects by administering therapeutic intestinal alkaline phosphatases. The present disclosure further relates to compositions comprising the combination of immune-checkpoint immunotherapies with therapeutic alkaline phosphatases and use of the compositions in the prevention and/or treatment of immune checkpoint inhibitor-mediated gastrointestinal side effects, such as colitis.

### CROSS-REFERENCE TO RELATED APPLICATIONS

This application claims the benefit of U.S. Provisional Application No. 63/396,398, filed Aug. 9, 2022, the entire contents of which are hereby incorporated by reference in their entirety.

### TECHNICAL FIELD

The present disclosure relates, inter alia, to methods for the prevention and/or reduction of immune checkpoint immunotherapy-mediated gastrointestinal side effects by administering therapeutic intestinal alkaline phosphatases.

### DESCRIPTION OF THE XML FILE SUBMITTED ELECTRONICALLY

The instant application contains a Sequence Listing which has been submitted electronically in XML file format and is hereby incorporated by reference in its entirety. Said XML file, created on Aug. 1, 2023, is named “SYN-060_Sequence_Listing.xml” and is 28,615 bytes in size.

### BACKGROUND

Administration of immune checkpoint inhibitors (CPI) has revolutionized the field of cancer treatment as these therapies provide improved efficacy as compared to standard cancer treatment, such as chemotherapy and radiation therapy. However, CPI treatment can be limiting due to the onset of gastrointestinal (GI) distress that accompanies CPI treatment, such as diarrhea and/or colitis.

Alkaline phosphatase (“APs,” EC 3.1.3.1) is a hydrolase enzyme that can remove phosphate groups from various targets, including nucleotides and proteins. In particular, mammalian APs exert their properties by primarily targeting LPS (a TLR4 agonist), flagellin (a TLR5 agonist) and CpG DNA (a TLR9 agonist). APs also degrade intestine luminal NTPs (e.g., ATP, GTP, etc.), which promote the growth of good bacteria and reverses dysbiosis. Accordingly, APs may find clinical use in, for example, treating various GI disorders.

Given the need for improved cancer therapies and mitigation of harmful side effects to allow for improved cancer patient care, there is a growing need for therapeutic compositions that prevent and/or reduce the side effects associated with CPIs.

### SUMMARY

Accordingly, in some aspects, the present disclosure provides methods for preventing or reducing CPI-mediated GI side effects in patients that experience such side effects, such as cancer patients, by administering one or more intestinal alkaline phosphatases (IAPs) constructs, including variants thereof. In embodiments, the IAP construct is a mammalian IAP including, but not limited to, human IAP (hIAP), calf IAP (cIAP), and bovine IAP (bIAP). In embodiments, the IAP is secreted from the host cell. In embodiments, the IAP and is administered orally. In embodiments, the IAP is administered before the CPI.

In some aspects, the patient is undergoing therapy with an immune checkpoint inhibitor immunotherapy, such as an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

In some aspects, the present disclosure provides methods for improving and/or increasing efficacy of an immune checkpoint inhibitor immunotherapy (e.g., used in tumor treatment). For example, in embodiments, the methods of the present disclosure improve efficacy of the CPI by increasing the therapeutic window of the CPI. In embodiments, the efficacy of a CPI therapy is not hindered by administration of an IAP construct. In embodiments, administration of an IAP construct does not decrease efficacy of a CPI therapy. In further embodiments, administration of an IAP construct improves anti-tumor efficacy associated with a CPI therapy.

In another aspect, the present disclosure provides methods for preventing CPI-mediated GI side effects by administering therapeutic IAPs and an additional therapeutic agent, such as, but not limited to, compositions comprising immune checkpoint immunotherapies selected from an agent that modulates one or more of PD-1, PD-L1, PD-L2, ICOS, ICOSL, and CTLA-4.

In another aspect, the present disclosure provides methods for treating cancer by administering therapeutic IAPs and an additional therapeutic agent, such as, but not limited to, compositions comprising immune checkpoint immunotherapies selected from an agent that modulates one or more of PD-1, PD-L1, PD-L2, ICOS, ICOSL, and CTLA-4, where the treatment has less CPI-mediated GI side effects than treatment in the absence of IAP.

In another aspect, the present disclosure provides methods for the therapeutic use of an IAP. In some aspects, an IAP is administered to a patient undergoing therapy with an immune checkpoint inhibitor immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4). In other aspects, a therapeutically effective amount of a composition comprising an immune checkpoint inhibitor immunotherapy is administered to a patient undergoing therapy with IAP.

In embodiments, the present disclosure provides methods for the prevention or treatment of a CPI-mediated GI side effect. In certain embodiments, the present disclosure provides methods for the treatment of a CPI-mediated GI side effect such as diarrhea and/or colitis. In embodiments, the present disclosure provides for a patient that is undergoing treatment with corticosteroids and/or agents targeting TNF-α for mediated GI side effect treatments. In embodiments, the present disclosure provides methods for reducing and/or obviating the dose or frequency of administration of corticosteroids and/or agents targeting TNF-α needed for mediated GI side effect treatments.

### BRIEF DESCRIPTION OF THE DRAWINGS

FIG. 1 depicts sequences pertaining to alkaline phosphatase agents present in compositions and/or formulations described herein.

### DETAILED DESCRIPTION

The present disclosure is based, inter alia, on the discovery that alkaline phosphatases, such as IAP, find use in preventing, reducing, or eliminating CPI-mediated GI side effects, such as diarrhea and/or colitis, in patients. Such use of alkaline phosphatases (e.g. IAP) allows for more efficacious cancer treatment as CPI therapy is not limited by dose- and regimen-limiting side effects of the GI. In embodiments, use of alkaline phosphatases improve efficacy (e.g., anti-tumor efficacy) of CPI therapies. Further, the present discovery includes methods in which patients are spared steroidal treatments that are currently used to reduce CPI-mediated GI side effects.

Cancer immunotherapy involves the utilization of naturally derived or synthetically generated components to stimulate or enhance the immune system to fight cancer. Immune checkpoint inhibitor immunotherapies are effective in fighting cancer due to the priming and activation of the immune system in order to produce antitumor effects, often involving highly specific targeting. Along with the promise of cancer immunotherapy, there is also the need to maintain the immune system's complex counterbalance between identification and eradication of foreign antigens and the processes necessary for suppressing an uncontrolled immune response. Indeed, there is a need to mitigate potentially harmful side effects that are associated with cancer immunotherapies, such as immune checkpoint inhibitor immunotherapies. Such side effects can include, but are not limited to, diarrhea and/or colitis. Common practice today involves minimizing checkpoint inhibition side effects, or immune-related adverse events, by interrupting the checkpoint inhibitor immunotherapy and administering agents that temporarily induce immunosuppression, such as, but not limited to, corticosteroids and/or tumor necrosis factor-alpha (TNF-α) antagonists.

IAP is an endogenous protein expressed by the intestinal epithelium that can be used to mitigate inflammation and maintain gut homeostasis. For example, loss of IAP expression or function is associated with increased intestinal inflammation, dysbiosis, bacterial translocation, and systemic inflammation. Its primary functions, among others, in maintaining intestinal homeostasis are generally recognized as the regulation of bicarbonate secretion and duodenal surface pH, long chain fatty acid absorption, mitigation of intestinal inflammation through detoxification of pathogen-associated molecular patterns, and regulation of the gut microbiome. Several substrates that are acted on by IAP's phosphatase functions include lipopolysaccharide (LPS), flagellin, CpG DNA, and nucleotide di- and tri-phosphates. Specifically, IAP is a target for therapeutics due to its ability to downregulate inflammation, regulate the microbiome, tighten the gut barrier through enhanced expression of claudins and occludins, and affect metabolism of adenosine tri-phosphate and diphosphate (ATP and ADP). In one aspect, the present disclosure provides a composition comprising IAP that does not hinder cancer treatment to the patient. In fact, according to the present disclosure, the methods described herein increase a therapeutic window of the CPI.

The present disclosure is directed, in part, to pharmaceutical compositions, formulations, and uses of a combination of one or more IAPs and a composition comprising one or more CPIs, including, but not limited to, one or more agents that modulates one or more of PD-1, PD-L1, PD-L2, ICOS, ICOSL, and CTLA-4. In certain aspects, the patient is undergoing therapy with a composition comprising an immune checkpoint inhibitor that includes, but is not limited to, one or more agents that modulates one or more of PD-1, PD-L1, PD-L2, ICOS, ICOSL, and CTLA-4.

### Alkaline Phosphatases (APs)

The present disclosure is directed, in part, to pharmaceutical compositions, formulations, and uses of one or more alkaline phosphatases. Alkaline phosphatases are dimeric metalloenzymes that catalyze the hydrolysis of phosphate esters and dephosphorylate a variety of target substrates at physiological and higher pHs. Illustrative APs that may be utilized in the present disclosure include, but are not limited to, IAP (e.g., calf IAP or bovine IAP, chicken IAP, goat IAP), placental alkaline phosphatase (PLAP), placental-like alkaline phosphatase, germ cell alkaline phosphatase (GCAP), tissue non-specific alkaline phosphatase (TNAP; which is primarily found in the liver, kidney, and bone), bone alkaline phosphatase, liver alkaline phosphatase, kidney alkaline phosphatase, bacterial alkaline phosphatase, fungal alkaline phosphatase, shrimp alkaline phosphatase, modified IAP, recombinant IAP, or any polypeptide comprising alkaline phosphatase activity.

In embodiments, the present disclosure contemplates the use of mammalian alkaline phosphatases including, but are not limited to, IAP, placental alkaline phosphatase (PLAP), germ cell alkaline phosphatase (GCAP), and the tissue non-specific alkaline phosphatase (TNAP).

### IAP

In embodiments, the alkaline phosphatase is IAP. IAP is produced in the proximal small intestine and is bound to the enterocytes via a glycosyl phosphatidylinositol (GPI) anchor. Some IAP is released into the intestinal lumen in conjunction with vesicles shed by the cells and as soluble protein stripped from the cells via phospholipases. The enzyme then traverses the small and large intestine such that some active enzyme can be detected in the feces. In embodiments, the IAP is human IAP (hIAP). In embodiments, the IAP is calf IAP (cIAP), also known as bovine IAP (bIAP). There are multiple isozymes of bIAP, for example, with bIAP II and IV having higher specific activity than bIAP I. In embodiments, the IAP is any one of the cIAP or bIAP isozymes (e.g., bIAP I, II, and IV). In embodiments, the IAP is bIAP II. In embodiments, the IAP is bIAP IV.

In embodiments, the IAP of the present disclosure has greater specific enzymatic activity than commercially-available APs, e.g., calf IAP (cIAP). In a non-limiting embodiment, the IAP of the present disclosure has specific enzymatic activity about 1-fold, 2-fold, 5-fold, 10-fold, 50-fold, 100-fold, 500-fold, 1000-fold, 10,000-fold, 100,000-fold, or 1,000,000-fold greater than the specific enzymatic activity of commercially-available APs, e.g., calf IAP (cIAP).

### IAP Variants

Also included within the definition of IAPs are IAP variants. An IAP variant has at least one or more amino acid modifications, generally amino acid substitutions, as compared to the parental wild-type sequence. In embodiments, an IAP of the disclosure comprises an amino sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein. In addition, IAP variants retain most or all of their biochemical activity, measured as described herein. In embodiments, the IAP comprises an amino acid sequence having at least about 60%, 65%, 70%, 75%, 80%, 85%, 90%, 95%, 96%, 97%, 98%, 99%, or 100% identity with any one of SEQ ID NOs: 1-6, or 10-14. In embodiments, the IAP comprises an amino acid sequence having at least about 60%, 65%, 70%, 75%, 80%, 85%, 90%, 95%, 96%, 97%, 98%, 99%, or 100% identity with SEQ ID NO: 11.

### GPI Anchored Proteins

Mammalian alkaline phosphatases are GPI anchored proteins. They have signal peptides and are translated into the secretory pathway. Once in the endoplasmic reticulum (ER), the proteins are glycosylated and folded. There are two disulfide bonds as well as a single free cysteine that is apparently not accessible on the surface. In the late ER, the carboxy terminus is removed and the GPI anchor is appended. GPI anchoring is therefore a process that occurs at the carboxy terminus of the alkaline phosphatase. The inclusion of stop codons at the anchor site enables secretion of biologically active protein (presumably the homodimer). While there is no consensus sequence, the carboxy terminus includes three amino acids, termed omega, omega +1, and omega +2 which are followed by a short stretch of hydrophilic amino acids and then a stretch of hydrophobic amino acids. Without wishing to be bound by theory, it is believed that the hydrophobicity is critical for embedding the carboxy terminus in the ER membrane. There, an enzymatic reaction replaces the carboxy terminus with the GPI anchor.

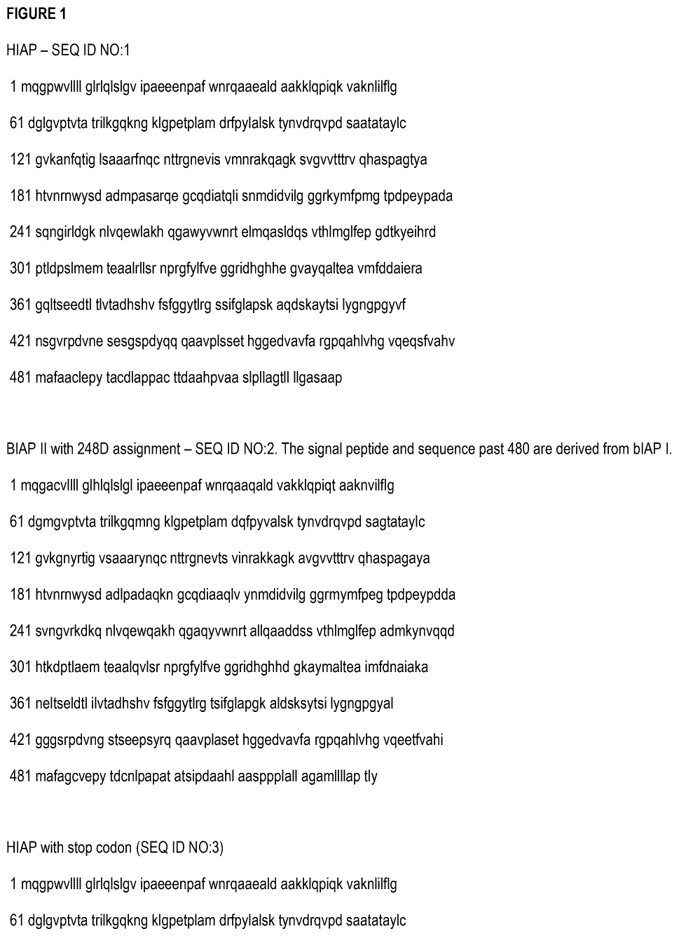

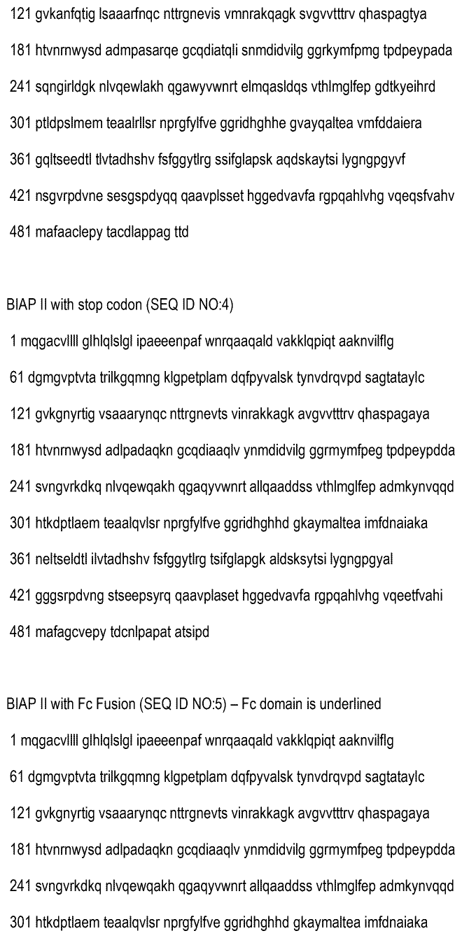

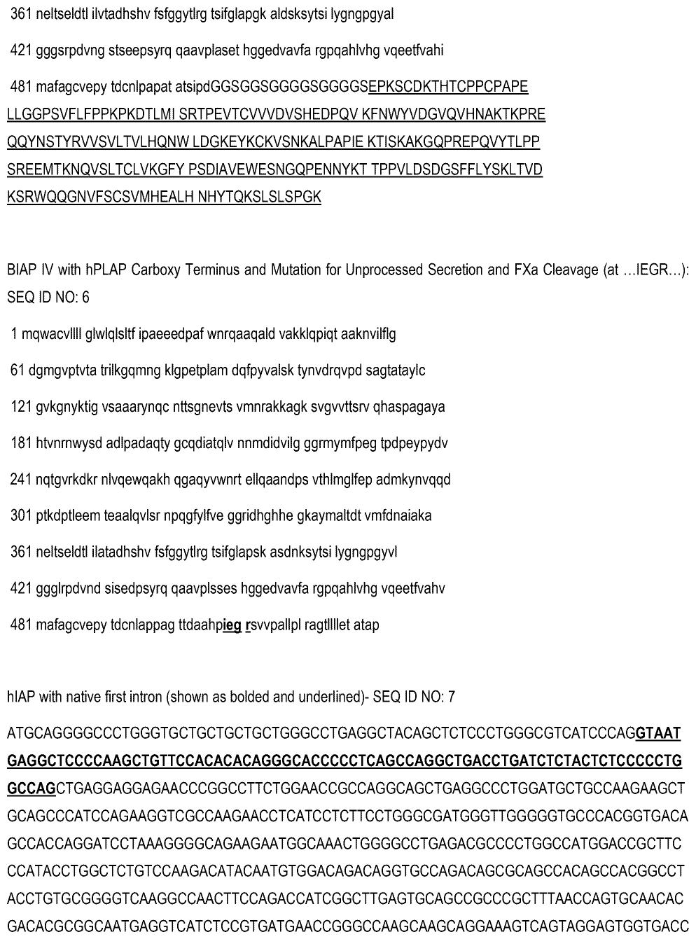

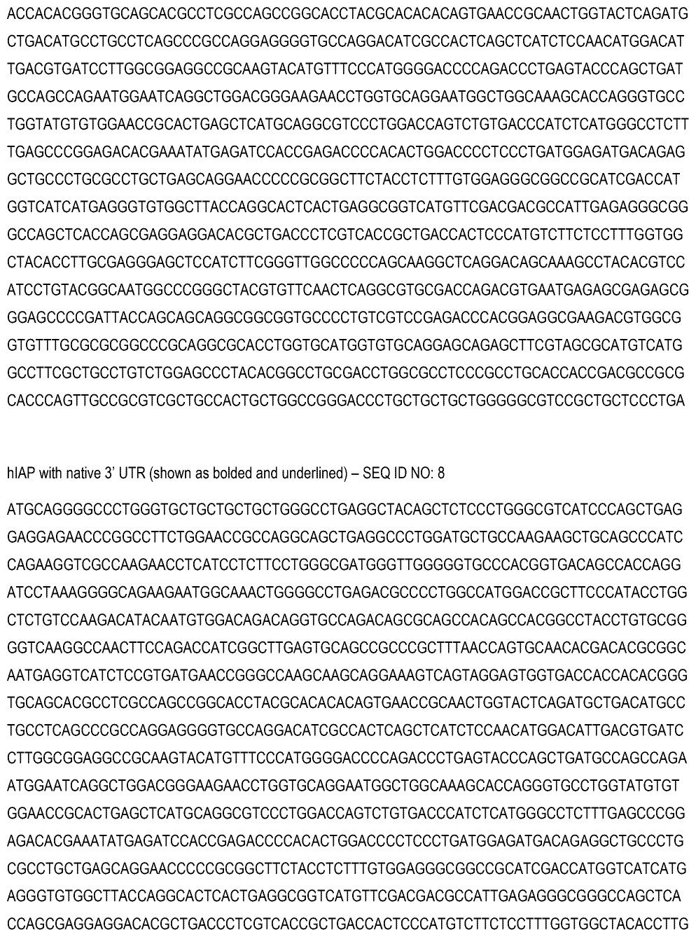

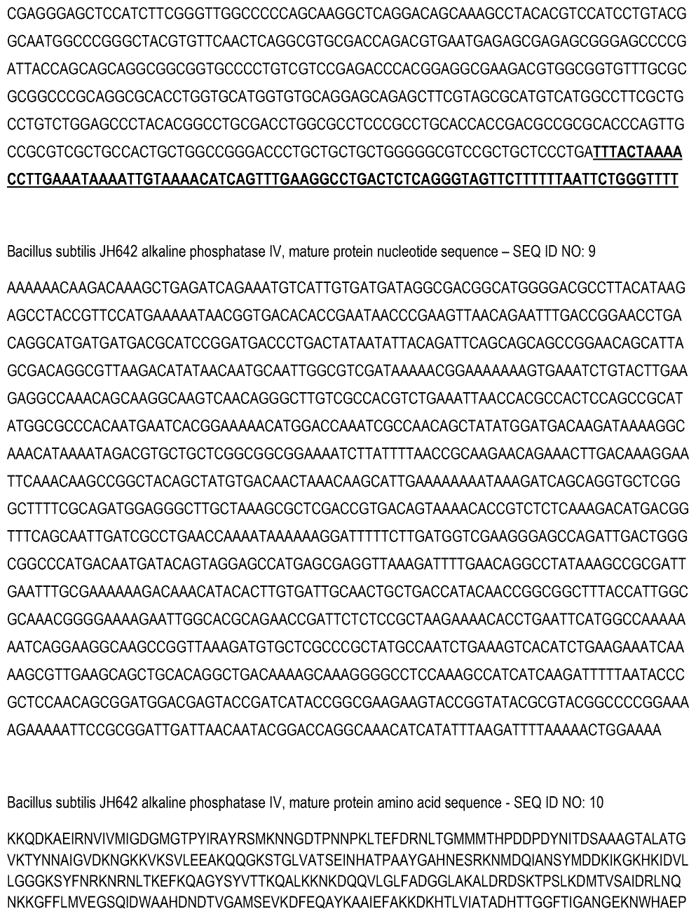

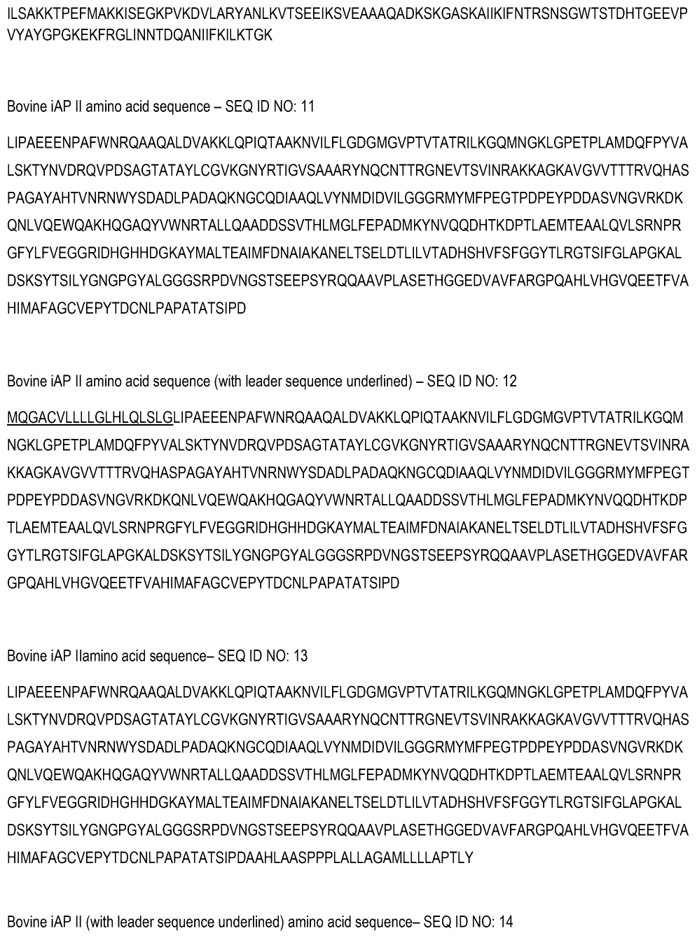

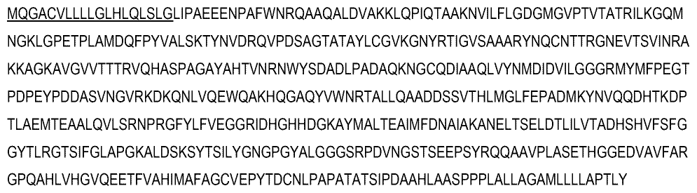

In embodiments, the IAP of the disclosure is a secreted protein; that is, in embodiments, the IAP is not GPI anchored, leading to secretion rather than intracellular retention. This can be accomplished in several ways. In embodiments, the IAP may lack the GPI anchor site, e.g. have the DAAH site removed, leading to secretion. Alternatively, this can be accomplished in embodiments, the IAP comprises a stop codon that is inserted immediately before the GPI anchor site. In embodiments, the IAP comprises a stop codon after the aspartate in the DAAH consensus site (e.g., at amino acid 503 of hIAP and bIAP IV or amino acid 506 of bIAP II). FIG. 1 depicts HIAP with a stop codon (SEQ ID NO:3) and bIAP II with a stop codon (SEQ ID NO:4).

### Human IAP

In embodiments, the IAP is human IAP (hIAP). In embodiments, the IAP is hIAP comprising the amino acid sequence of SEQ ID NO:1 as depicted in FIG. 1 or a variant as described herein, as long as the hIAP variant retains at least 75, 80, 85, 90, 95, 96, 97, 98, 99 or 100% of the phosphatase activity as compared to the wild type enzyme using an assay as outlined herein.

Included within the definition of hIAP are amino acid modifications, with amino acid substitutions finding particular use in the present disclosure. For example, without wishing to be bound by theory, it is believed that a cysteine at the carboxy terminus of the AP-based agent (e.g., at position 500 of SEQ ID NO:1) may interfere with protein folding. Accordingly, in embodiments, the AP-based agent includes a mutation of the cysteine (e.g., at position 500 of SEQ ID NO: 1). In embodiments, the cysteine is replaced with any amino acid, although glycine finds particular use in embodiments. Furthermore, the C-terminal cysteine can also be deleted.

In embodiments, the hIAP is a recombinant, glycosylated hIAP. In embodiments, the hIAP is glycosylated with one or more terminal sialic acids. In embodiments, the recombinant hIAP is glycosylated on the N-terminus, C-terminus, and/or is glycosylated on one or more residues located internally within the primary amino acid sequence. In embodiments, the recombinant hIAP is terminally sialylated, e.g., where the sialyation is present on the terminus of one or more glycosylation structures.

As will be appreciated by those in the art, additional amino acid modifications can be made in hIAP as discussed herein. For example, in embodiments, a stop codon may be inserted after the aspartate in the DAAH consensus site (e.g., at amino acid 503 of hIAP). FIG. 1 depicts hIAP with an inserted stop codon (SEQ ID NO:3).

### Fusion Proteins

In embodiments, the present disclosure provides for chimeric proteins. In embodiments, the present disclosure provides for chimeric fusion proteins. For example, in embodiments, the present disclosure provides an isolated or recombinant alkaline phosphatase comprising a crown domain and a catalytic domain, wherein said crown domain and said catalytic domain are obtained from different alkaline phosphatases (e.g., human and bovine alkaline phosphatases). In embodiments, the alkaline phosphatases are both human APs. In certain embodiments, the present disclosure provides for recombinant fusion proteins comprising human IAP and a domains of human placental alkaline phosphatases. In certain embodiments, the present disclosure provides for chimeric hIAP-placenta fusion proteins. In embodiments, the IAP is a human recombinant fusion protein, such as a human placental AP/intestinal AP fusion protein (e.g., llofotase alfa).

In embodiments, the AP-based agent of the disclosure is a fusion protein. In embodiments, the AP-based agent comprises an alkaline phosphatase fused to a protein domain that replaces the GPI anchor sequence. In embodiments, the alkaline phosphatase is fused to a protein domain that promotes protein folding and/or protein purification and/or protein dimerization and/or protein stability. In embodiments, the AP-based agent fusion protein has an extended serum half-life.

In embodiments, the alkaline phosphatase is fused to an immunoglobulin Fc domain and/or hinge region. In embodiments, the immunoglobulin Fc domain and/or hinge region is derived from the Fc domain and/or hinge region of an antibody (e.g., of IgG, IgA, IgD, and IgE, inclusive of subclasses (e.g. IgG1, IgG2, IgG3, and IgG4, and IgA1 and IgA2). In embodiments, the AP-based agent of the disclosure comprises an alkaline phosphatase fused to the hinge region and/or Fc domain of IgG.

In embodiments, the AP-based agent of the disclosure is a pro-enzyme. In embodiments, the activity of the proenzyme is suppressed by a carboxy terminus. In embodiments, protease removal of the carboxy terminus reactivates the enzymatic activity of the alkaline phosphatase. In embodiments, the pro-enzyme is more efficiently secreted than the enzyme without the carboxy terminus.

In embodiments, for generation of the pro-enzyme, the native carboxy terminus of the alkaline phosphatase is replaced with the analogous sequence from hPLAP. In embodiments, a mutation is made in the hydrophobic carboxy tail to promote protein secretion without cleavage of the carboxy terminus. In embodiments, a single point mutation such as a substitution of leucine with e.g., arginine is generated in the hydrophobic carboxy terminus (e.g. allpllagtl is changed to e.g., allplragtl, e.g., Leu515 is changed to e.g., Arg515 in SEQ ID NO: 1) to result in secretion of the enzyme without removal of the carboxy terminus.

### Bovine IAPs

In embodiments, the IAP is bovine IAP (bIAP). In embodiments, the bIAP is selected from bIAP I, bIAP II, and bIAP IV.

In embodiments, the IAP comprises an amino sequence having at least about 90%, or about 95%, or about 97%, or about 98%, or about 99% sequence identity with any one of SEQ ID NOs: 1-14. In embodiments, the IAP comprises an amino sequence having at least about 97% sequence identity to SEQ ID NO: 11. In embodiments, the IAP comprises an amino sequence having at least about 99% sequence identity to SEQ ID NO: 11.

Wild-type bIAP produced in calf intestine is not naturally sialylated. In embodiments, the bIAP is a recombinant, sialylated bIAP. In embodiments, the bIAP is a recombinant, glycosylated bIAP. In embodiments, the bIAP is glycosylated with one or more terminal sialic acids. In embodiments, the recombinant bIAP is glycosylated on the N-terminus, C-terminus, and/or is glycosylated on one or more residues located internally within the primary amino acid sequence. In embodiments, the recombinant bIAP is terminally sialylated, e.g., where the sialyation is present on the terminus of one or more glycosylation structures.

In embodiments, the recombinant, sialylated bIAP comprises an amino sequence having about or at least about 90%, about or at least about 95%, about or at least about 96%, about or at least about 97%, about or at least about 98%, or about or at least about 99% or more sequence identity to SEQ ID NO: 11. In embodiments, the recombinant, sialylated bIAP comprises an amino sequence of SEQ ID NO: 11.

In embodiments, the recombinant IAP (e.g., sialylated bIAP) of the present disclosure has substantially equal or greater specific activity than commercially available APs, e.g., calf IAP (cIAP). In embodiments, the recombinant IAP (e.g., sialylated bIAP) of the present disclosure has substantially equal or greater half-life than commercially available IAPs.

a. bIAP II

In embodiments, the IAP is bovine IAP II (bIAP II) or a variant as described herein, as long as the bIAP variant retains at least 75, 80, 85, 90, 95, 96, 97, 98, 99 or 100% of the phosphatase activity using an assay as outlined herein. In embodiments, the bIAP II comprises the signal peptide and carboxy terminus of bIAP I. In embodiments, the bIAP II comprises an aspartate at position 248 (similar to bIAP IV). In embodiments, the bIAP II comprises the amino acid sequence of SEQ ID NO:2, or a sequence having at least about 80%, 85%, 90%, 95%, 96%, 97%, 98%, or 99% sequence identity thereto. FIG. 1 depicts BIAP II with 248D assignment-SEQ ID NO:2. The signal peptide and sequence past 480 are derived from bIAP I.

In embodiments, the bIAP II comprises SEQ ID NO: 11, or a sequence having at least about 80%, 85%, 90%, 95%, 96%, 97%, 98%, or 99% sequence identity thereto. In embodiments, the bIAP II comprises, consists of, or consists essentially of the amino acid sequence of SEQ ID NO: 11.

Also included within the definition of bIAP II are amino acid variants as described herein. For example, in embodiments, a stop codon may be inserted after the aspartate in the DAAH consensus site (e.g., at amino acid 506 of bIAP II). FIG. 1 depicts bIAP II with an inserted stop codon (SEQ ID NO:4).

### Expression Variants

In embodiments, the IAP of the disclosure is efficiently expressed and secreted from a host cell. In embodiments, the IAP of the disclosure is efficiently transcribed in a host cell. In embodiments, the IAP exhibits enhanced RNA stability and/or transport in a host cell. In embodiments, the IAP is efficiently translated in a host cell. In embodiments, the IAP exhibits enhanced protein stability.

In embodiments, the IAPs are efficiently expressed in a host cell. In embodiments, the Kozak sequence of the DNA construct encoding the AP-based agent is optimized. The Kozak sequence is the nucleotide sequence flanking the ATG start codon that instructs the ribosome to start translation. There is flexibility in the design of a Kozak sequence, but one canonical sequence is GCCGCCACCATGG (SEQ ID NO: 15). The purine in the −3 position and the G in the +4 position are the most important bases for translation initiation. For hIAP, bIAP II, and bIAP IV, the second amino acid, that is, the one after the initiator methionine, is glutamine. Codons for glutamine all have a C in the first position. Thus, their Kozak sequences all have an ATGC sequence. Accordingly, in embodiments, the ATGC sequence is changed to ATGG. This can be achieved by changing the second amino acid to a glycine, alanine, valine, aspartate, or glutamic acid, all of whose codons have a G in the first position. These amino acids may be compatible with signal peptide function. In alternative embodiments, the entire signal peptide is substituted for peptide having a canonical Kozak sequence and is derived from a highly expressed protein such as an immunoglobulin.

In embodiments, the signal peptide of the IAP may be deleted and/or substituted. For example, the signal peptide may be deleted, mutated, and/or substituted (e.g., with another signal peptide) to ensure optimal protein expression.

In embodiments, the DNA construct encoding the IAP of the disclosure comprises untranslated DNA sequences. Such sequences include an intron, which may be heterologous to the IAP protein or native to the IAP protein including the native first and/or second intron and/or a native 3′ UTR. Without wishing to be bound by theory, it is believed that include of these sequences enhance protein expression by stabilizing the mRNA. Accordingly, in embodiments, the DNA construct encoding the IAP of the disclosure comprises the 5′UTR and/or the 3′UTR. Provided in FIG. 1 are illustrative IAP DNA sequences with a first intron and a 3′UTR, including hIAP with native first intron (shown as bolded and underlined)—SEQ ID NO: 7; and hIAP with native 3′ UTR (shown as bolded and underlined)—SEQ ID NO: 8.

In embodiments, the IAP of the disclosure is encoded by a nucleotide sequence having at least about 60% (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99%) sequence identity with any of the sequences disclosed herein.

In embodiments, the IAP of the disclosure may comprise an amino acid sequence having one or more amino acid mutations relative to any of the protein sequences described herein. In embodiments, the one or more amino acid mutations may be independently selected from substitutions, insertions, deletions, and truncations.

In embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β-methyl amino acids, C α-methyl amino acids, N α-methyl amino acids, and amino acid analogs in general).

Mutations may be made to the IAP of the disclosure to select for agents with desired characteristics. For examples, mutations may be made to generate IAPs with enhanced catalytic activity or protein stability. In embodiments, directed evolution may be utilized to generate IAPs of the disclosure. For example, error-prone PCR and DNA shuffling may be used to identify mutations in the bacterial alkaline phosphatases that confer enhanced activity.

### Immune Checkpoint Inhibitor (CPI) Immunotherapy

The present disclosure provides, in part, pharmaceutical compositions, formulations, and uses of immune checkpoint inhibitor immunotherapies. Cancer immunotherapy involves the utilization of naturally derived or synthetically generated components to stimulate or enhance the immune system to fight cancer. Immune checkpoint inhibitor immunotherapies are effective in fighting cancer due to the priming and activation of the immune system in order to produce antitumor effects, often involving highly specific targeting. Along with the promise of cancer immunotherapy, there is the need to maintain the immune system's complex counterbalance between identification and eradication of foreign antigens and the processes necessary for suppressing an uncontrolled immune response. Despite important clinical benefits, checkpoint inhibition is associated with a unique spectrum of side effects, or immune-related adverse events, including, but not limited to, dermatologic, GI, hepatic, endocrine, and other less common inflammatory events. In various embodiments of the present disclosure, the CPI-mediated GI side effect is diarrhea and/or colitis. Generally, treatment of these moderate or severe immune checkpoint inhibitor immunotherapy-mediated side effects can require interruption of the checkpoint inhibitor immunotherapy and use of corticosteroid immunosuppression

In some aspects, the present disclosure contemplates methods for improving and/or increasing and/or enhancing efficacy of an immune checkpoint inhibitor immunotherapy (e.g., used in tumor treatment). For example, in embodiments, the methods of the present disclosure improve efficacy of the CPI by increasing the therapeutic window of the CPI. In embodiments, the efficacy of a CPI therapy is not hindered by administration of an IAP construct. In embodiments, administration of an IAP construct does not decrease efficacy of a CPI therapy. In further embodiments, administration of an IAP construct improves anti-tumor efficacy associated with a CPI therapy. In embodiments of the present disclosure, the efficacy of an immune checkpoint inhibitor immunotherapy is enhanced, for example, as measured by the immunotherapies' increased antitumor effects.

In some aspects, the present disclosure provides pharmaceutical compositions, formulations, and uses of immune checkpoint inhibitor immunotherapies. Such CPIs can include, but are not limited to, one or more agents that modulate one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4). In embodiments, the patient is undergoing therapy with an immune checkpoint inhibitor immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

In other aspects, the present disclosure contemplates methods for preventing CPI-mediated GI side effects by administering a combination of an IAP and an CPI selected from an agent that modulates one or more of PD-1, PD-L1, PD-L2, ICOS, ICOSL, and CTLA-4.

In embodiments, the agent that modulates one or more of PD-1, PD-L1, PD-L2, ICOS, ICOSL, and CTLA-4 is an antibody or antibody format specific for one or more of PD-1, PD-L1, PD-L2, ICOS, ICOSL, and CTLA-4. In embodiments, the antibody or antibody format specific for one or more of PD-1, PD-L1, PD-L2, ICOS, ICOSL, and CTLA-4 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

For example, in embodiments, the present disclosure provides for an CPI that is an agent that modulates PD-1, wherein the agent is an antibody or antibody format specific for PD-1. In embodiments, the antibody or antibody format specific for PD-1 is selected from Nivolumab, Pembrolizumab, and Pidilizumab. In other embodiments, the present disclosure provides for an CPI that is an agent that modulates PD-L1, wherein the agent is an antibody or antibody format specific for PD-L1. In embodiments, the antibody or antibody format specific for PD-L1 is selected from BMS-936559, Atezolizumab, Avelumab and Durvalumab. In other embodiments, the present disclosure provides for an CPI that is an agent that modulates PD-L2, wherein the agent is an antibody or antibody format specific for PD-L2. In other embodiments, the present disclosure provides for an CPI that is an agent that modulates ICOS, wherein the agent is an antibody or antibody format specific for ICOS. In embodiments, the antibody or antibody format specific for ICOS comprises JTX-2011. In other embodiments, the present disclosure provides for an CPI that is an agent that modulates ICOSL, wherein the agent is an antibody or antibody format specific for ICOSL. In other embodiments, the present disclosure provides for an CPI that is an agent that modulates CTLA-4, wherein the agent is an antibody or antibody format specific for CTLA-4. In embodiments, the antibody or antibody format specific for CTLA-4 is selected from tremelimumab or Ipilimumab.

### Additional Therapeutics for Treating Immune Checkpoint Inhibitor-Mediated GI Side Effects

The present disclosure provides, in part, methods for preventing and/or reducing CPI-mediated GI side effects by further administering to a patient a corticosteroid and/or an agent targeting tumor necrosis factor alpha (TNF-α) in order to provide temporary immunosuppression. In embodiments, the patient is undergoing therapy with one or more corticosteroids. In embodiments, the patient is undergoing therapy with one or more agents targeting TNF-α. In embodiments, the one or more agents targeting TNF-α is an antibody or a fusion protein. In embodiments, the one or more agents targeting TNF-α is selected from infliximab (Remicade), infliximab-dyyb (Inflectra), infliximab-abda (Renflexis) or Flixabi. In embodiments, administering a corticosteroid and/or an agent targeting TNF-α to a patient undergoing CPI interrupts the immunotherapy.

In embodiments, the present disclosure provides for methods of preventing and/or reducing an CPI-mediated GI side effect by administering IAP to a patient that is further undergoing treatment with a corticosteroid and/or an agent targeting TNF-α. In embodiments, the IAP treatment reduces the dose or frequency of corticosteroid administration needed for immunotherapy-mediated GI side effect treatment. In embodiments, the IAP treatment obviates corticosteroid administration for immunotherapy-mediated GI side effect treatment. In embodiments, the IAP treatment reduces the dose or frequency of administration of one or more agents targeting TNF-α needed for immunotherapy-mediated GI side effect treatment. In embodiments, the IAP treatment obviates administration of one or more agents targeting TNF-α for immunotherapy-mediated GI side effect treatment.

In embodiments, the patient is a cancer patient. In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including GI cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

### Methods of Making IAP of the Disclosure

The IAPs of the disclosure are made using standard molecular biology techniques. For example, nucleic acid compositions encoding the IAPs of the disclosure are also provided, as well as expression vectors containing the nucleic acids and host cells transformed with the nucleic acid and/or expression vector compositions. As will be appreciated by those in the art, the protein sequences depicted herein can be encoded by any number of possible nucleic acid sequences, due to the degeneracy of the genetic code.

As is known in the art, the nucleic acids encoding the components of the disclosure can be incorporated into expression vectors as is known in the art, and depending on the host cells, used to produce the IAP compositions of the disclosure. Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the disclosure are then transformed into any number of different types of host cells as is well known in the art, including mammalian, bacterial, yeast, insect and/or fungal cells, with mammalian cells (e.g. CHO cells), finding use in many embodiments.

In embodiments, the cell is a mammalian cell. In embodiments, the mammalian cell is a human cell. In embodiments, the cell is an insect cell. In embodiments, the cell is immortalized.

In embodiments, the cell is a Chinese hamster ovary (CHO), baby hamster kidney (BHK), human embryonic kidney (HEK293T) cells, Vero cell, or Spodoptera frugiperda 9 (Sf9) cell. In embodiments, the CHO cell is a CHO-K1, CHO-DHB11, CHO-DXB1, CHO-S, or CHO-DG44 cell. In embodiments, a CHO cell comprises or is selected from CHO-K1 (ATCC CCL-61) cells, SURE CHO-M cells (derivative of CHO-K1), or baby hamster kidney cells (BHK, ATCC CCL-10). In embodiments, the Vero cell comprises or is selected from Vero, Vero 76 and Vero E6. In embodiments, the cell is a Per C6 cell line, e.g., a human embryonic retinal cell line transformed with the Adenovirus Type 5 (Ad5) E1A and E1B genes. In embodiments, the cell is an immortalized cell line based on primary human amniocytes (e.g., including amniotic fluid stem cells, somatic fetal stem cells), for example as generated by transfection with a vector containing the functions of E1 and pIX of Adenovirus Type 5 (Ad5) as done in CAP cell lines (CEVEC Pharmaceuticals, amniocyte production cell line).

In embodiments, the cell is, without limitations, human cervical carcinoma cells (HELA, ATCC CCL-2), 293 (ATCC CRL-1573), 3T3 (ATCC CCL-163), or monkey kidney CV1 line (ATCC CCL-70), which can be transformed with SV40 (COS-7, ATCC CRL-1587).

The IAPs of the disclosure are made by culturing host cells comprising the expression vector(s) as is well known in the art. Once produced, traditional purification steps are done. In embodiments, IAPs of the disclosure are manufactured by i) introducing a nucleic acid (e.g., an expression vector) encoding one or more APs described herein into a host cell, ii) culturing the host cell under conditions suitable for expression (e.g., under a selection antibiotic); and iii) isolating the IAP (e.g., using a variety of purification techniques, such as affinity chromatography, size exclusion, etc.).

### Formulations

The present disclosure provides the described IAP and/or composition comprising one or more CPIs (and/or additional therapeutic agents) in various formulations. Any IAP and/or or composition comprising one or more CPIs (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use. In embodiments, the IAP of the present disclosure is formulated in a delayed release capsule.

In embodiments, the IAP described herein is formulated into compositions adapted for any mode of administration described herein.

In embodiments, the IAP is formulated to be substantially released in the GI tract. In embodiments, the IAP is formulated to be substantially released in the small intestine. In embodiments, the IAP is formulated to be substantially released in the large intestine. In embodiments, the IAP is formulated to not be substantially released systemically.

The formulations comprising the IAP and/or composition comprising one or more CPIs (and/or additional therapeutic agents) may conveniently be presented in unit dosage forms. For example, the dosage forms may be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In embodiments, the IAP and/or composition comprising one or more CPIs (and/or additional therapeutic agents) described herein are formulated as compositions adapted for a mode of administration described herein.

some In embodiments, the recombinant IAP comprises an amino sequence having about or at least about 90%, about or at least about 95%, about or at least about 96%, about or at least about 97%, about or at least about 98%, or about or at least about 99% or more sequence identity to any one of SEQ ID NOs: 1-14.

In embodiments, the formulation of the present invention is in the form of a capsule (e.g., a hard gelatin or HPMC capsule) comprising about 15 mg of the AP-based agent (e.g. IAP, or the other AP-based agent agents described herein, and variants thereof). The capsule includes a plurality of AP-based agent-containing pellets. In embodiments, the formulation of the present invention comprises at least one modified-release pellet, wherein each modified-release pellet comprises about 5-25% by weight AP-based agent (e.g. IAP, or the other AP-based agent agents described herein, and variants thereof). For example, the AP-based agent (e.g. IAP, or the other AP-based agent agents described herein, and variants thereof) may be present at about 5%, about 6%, about 7%, about 8%, about 9%, about 10%, about 12%, about 13%, about 14%, about 15%, about 16%, about 17%, about 18%, about 19%, about 20%, about 21%, about 22%, about 23%, about 24%, or about 25% by weight. In embodiments, the pellets (or each individual pellet) comprise about 45-65% by weight sucrose sphere. For example, the sucrose sphere may be present at about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, or about 65% by weight. In embodiments, the pellets (or each individual pellet) comprise about 20-40% by weight hydroxypropylcellulose (HPC). For example, the hydroxypropylcellulose may be present at about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight. In embodiments, the pellets (or each individual pellet) comprise about 0.2-2% by weight of buffer salt. The buffer salts may be selected from a Tris base, magnesium chloride, magnesium sulfate, zinc chloride and zinc sulfate. For example, the buffer salts may be present at about 0.2%, 0.3%, 0.4%, 0.5%, about 0.6%, about 0.7%, about 0.8%, about 0.9%, about 1.0%, about 1.1%, about 1.2%, about 1.3%, about 1.4%, about 1.5%, 1.6%, about 1.7%, about 1.8%, about 1.9%, or about 2.0% by weight. In embodiments, each modified-release pellet comprises about or at least about 5-25% (w/w) recombinant IAP, about or at least about 45-65% (w/w) sucrose sphere, about or at least about 20-40% (w/w) hydroxypropylcellulose, and about or at least about 0.2-2% (w/w) buffer. In embodiments, each modified-release pellet includes about 10-15% (w/w) recombinant IAP, about 55-60% (w/w) sucrose sphere, about 25-30% (w/w) hydroxypropylcellulose, and about 0.2-1% (w/w) buffer. In embodiments, each modified-release pellet includes about 15% (w/w) recombinant IAP, about 55% (w/w) sucrose sphere, about 30% (w/w) hydroxypropylcellulose, and about 0.5% (w/w) buffer. In embodiments, each modified-release pellet includes about 14.5% (w/w) recombinant IAP, about 56.2% (w/w) sucrose sphere, about 28.9% (w/w) hydroxypropylcellulose, and about 0.4% (w/w) buffer.

In embodiments, the pellets (or each individual pellet) comprise about 10-40% by weight EUDRAGIT L30 D-55. For example, the EUDRAGIT L30 D-55 may be present at about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight. In embodiments, the pellets (or each individual pellet) comprise about 0.5-11% by weight HTP-20. For example, the HTP-20 may be present at about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, or about 11.0% by weight.

In embodiments, the formulation of the present invention is in the form of a capsule (e.g., a hard gelatin or HPMC capsule) comprising about 15 mg of the AP-based agent (e.g. IAP, or the other AP-based agent agents described herein, and variants thereof). The capsule includes a plurality of enteric-coated AP-based agent-containing pellets. In embodiments, the capsule comprises about 5-15% by weight AP-based agent (e.g. IAP, or the other AP-based agent agents described herein, and variants thereof). For example, the AP-based agent may be present at about 5%, about 6%, about 7%, about 8%, about 9%, about 10%, about 12%, about 13%, about 14%, or about 15% by weight. In embodiments, the capsule comprises about 35-45% by weight sucrose sphere. For example, the sucrose sphere may be present at about 35%, about 36%, about 37%, about 38%, about 39%, about 40%, about 41%, about 42%, about 43%, about 44%, or about 45% by weight. In embodiments, the capsule comprises about 15-25% by weight hydroxypropylcellulose (HPC). For example, the HPC may be present at about 15%, about 16%, about 17%, about 18%, about 19%, about 20%, about 21%, about 22%, about 23%, about 24%, or about 25% by weight. In embodiments, the capsule comprises about 0.1-1.5% by weight of buffer salt In embodiments, the capsule comprises about 20-30% by weight enteric polymer (e.g., EUDRAGIT L30 D-55). For example, the enteric polymer may be present at about 20%, about 21%, about 22%, about 23%, about 24%, about 25%, about 26%, about 27%, about 28%, about 29%, or about 30% by weight. In embodiments, the capsule comprises about 1-10% by weight HTP-20 (e.g., PLASACRYL HTP 20). For example, the HTP-20 may be present at about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight.

In embodiments, the formulation of the present invention is in the form of a capsule (e.g., a hard gelatin or HPMC capsule) comprising about 15 mg of the AP-based agent (e.g. IAP, or the other AP-based agent agents described herein, and variants thereof). The capsule includes a plurality of enteric-coated AP-based agent-containing pellets. In such embodiments, the formulation comprises about 10% by weight AP-based agent (e.g. IAP, or the other AP-based agent agents described herein, and variants thereof); about 39% by weight sucrose sphere; about 20% by weight hydroxypropylcellulose (HPC); about 0.5% by weight of buffer salt; about 26% by weight enteric polymer (e.g., EUDRAGIT L30 D-55), and about 4.5% by weight HTP-20 (e.g., PLASACRYL HTP 20).

In embodiments, the formulation of the present invention is in the form of a capsule (e.g., a hard gelatin or HPMC capsule) comprising about 15 mg of the AP-based agent (e.g. IAP, or the other AP-based agent agents described herein, and variants thereof). The capsule includes a plurality of enteric-coated AP-based agent-containing pellets. In such embodiments, the formulation comprises about 10.0% by weight AP-based agent (e.g. IAP, or the other AP-based agent agents described herein, and variants thereof); about 38.9% by weight sucrose sphere; about 20.0% by weight hydroxypropylcellulose (HPC); about 0.3% by weight of buffer salt; about 26.3% by weight enteric polymer (e.g., EUDRAGIT L 30 D-55), and about 4.5% by weight HTP-20 (e.g., PLASACRYL HTP 20).

In embodiments, the formulation of the present invention is in the form of a capsule (e.g., a hard gelatin or HPMC capsule) comprising about 5 mg of the AP-based agent (e.g. IAP, or the other AP-based agent agents described herein, and variants thereof). The capsule includes a plurality of AP-based agent-containing pellets. In embodiments, the formulation of the present invention comprises at least one modified-release pellet, wherein each modified-release pellet comprises about 5-25% by weight AP-based agent (e.g. IAP, or the other AP-based agent agents described herein, and variants thereof). For example, the AP-based agent (e.g. IAP, or the other AP-based agent agents described herein, and variants thereof) may be present at about 5%, about 6%, about 7%, about 8%, about 9%, about 10%, about 12%, about 13%, about 14%, about 15%, about 16%, about 17%, about 18%, about 19%, about 20%, about 21%, about 22%, about 23%, about 24%, or about 25% by weight. In embodiments, the pellets (or each individual pellet) comprise about 45-65% by weight sucrose sphere. For example, the sucrose sphere may be present at about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, or about 65% by weight. In embodiments, the pellets (or each individual pellet) comprise about 20-40% by weight hydroxypropylcellulose (HPC). For example, the hydroxypropylcellulose may be present at about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight. In embodiments, the pellets (or each individual pellet) comprise about 0.2-2% by weight of buffer salt. The buffer salts may be selected from a Tris base, magnesium chloride, magnesium sulfate, zinc chloride and zinc sulfate. For example, the buffer salts may be present at about 0.2%, 0.3%, 0.4%, 0.5%, about 0.6%, about 0.7%, about 0.8%, about 0.9%, about 1.0%, about 1.1%, about 1.2%, about 1.3%, about 1.4%, about 1.5%, 1.6%, about 1.7%, about 1.8%, about 1.9%, or about 2.0% by weight. In embodiments, each modified-release pellet comprises about or at least about 5-25% (w/w) recombinant IAP, about or at least about 45-65% (w/w) sucrose sphere, about or at least about 20-40% (w/w) hydroxypropylcellulose, and about or at least about 0.2-2% (w/w) buffer. In embodiments, each modified-release pellet includes about 10-15% (w/w) recombinant IAP, about 55-60% (w/w) sucrose sphere, about 25-30% (w/w) hydroxypropylcellulose, and about 0.2-1% (w/w) buffer. In embodiments, each modified-release pellet includes about 15% (w/w) recombinant IAP, about 55% (w/w) sucrose sphere, about 30% (w/w) hydroxypropylcellulose, and about 0.5% (w/w) buffer. In embodiments, each modified-release pellet includes about 14.5% (w/w) recombinant IAP, about 56.2% (w/w) sucrose sphere, about 28.9% (w/w) hydroxypropylcellulose, and about 0.4% (w/w) buffer.

In embodiments, the pellets (or each individual pellet) comprise about 10-40% by weight EUDRAGIT L30 D-55. For example, the EUDRAGIT L30 D-55 may be present at about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight. In embodiments, the pellets (or each individual pellet) comprise about 0.5-11% by weight HTP-20. For example, the HTP-20 may be present at about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, or about 11.0% by weight.

In embodiments, the formulation of the present invention is in the form of a capsule (e.g., a hard gelatin or HPMC capsule) comprising about 5 mg of the AP-based agent (e.g. IAP, or the other AP-based agent agents described herein, and variants thereof). The capsule includes a plurality of enteric-coated AP-based agent-containing pellets. In embodiments, the capsule comprises about 5-15% by weight AP-based agent (e.g. IAP, or the other AP-based agent agents described herein, and variants thereof). For example, the AP-based agent may be present at about 5%, about 6%, about 7%, about 8%, about 9%, about 10%, about 12%, about 13%, about 14%, or about 15% by weight. In embodiments, the capsule comprises about 35-45% by weight sucrose sphere. For example, the sucrose sphere may be present at about 35%, about 36%, about 37%, about 38%, about 39%, about 40%, about 41%, about 42%, about 43%, about 44%, or about 45% by weight. In embodiments, the capsule comprises about 15-25% by weight hydroxypropylcellulose (HPC). For example, the HPC may be present at about 15%, about 16%, about 17%, about 18%, about 19%, about 20%, about 21%, about 22%, about 23%, about 24%, or about 25% by weight. In embodiments, the capsule comprises about 0.1-1.5% by weight of buffer salt In embodiments, the capsule comprises about 20-30% by weight enteric polymer (e.g., EUDRAGIT L30 D-55). For example, the enteric polymer may be present at about 20%, about 21%, about 22%, about 23%, about 24%, about 25%, about 26%, about 27%, about 28%, about 29%, or about 30% by weight. In embodiments, the capsule comprises about 1-10% by weight HTP-20 (e.g., PLASACRYL HTP 20). For example, the HTP-20 may be present at about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight.

In embodiments, the formulation of the present invention is in the form of a capsule (e.g., a hard gelatin or HPMC capsule) comprising about 5 mg of the AP-based agent (e.g. IAP, or the other AP-based agent agents described herein, and variants thereof). The capsule includes a plurality of enteric-coated AP-based agent-containing pellets. In such embodiments, the formulation comprises about 10% by weight AP-based agent (e.g. IAP, or the other AP-based agent agents described herein, and variants thereof); about 39% by weight sucrose sphere; about 20% by weight hydroxypropylcellulose (HPC); about 0.5% by weight of buffer salt; about 26% by weight enteric polymer (e.g., EUDRAGIT L30 D-55), and about 4.5% by weight HTP-20 (e.g., PLASACRYL HTP 20).

In embodiments, the formulation of the present invention is in the form of a capsule (e.g., a hard gelatin or HPMC capsule) comprising about 5 mg of the AP-based agent (e.g. IAP, or the other AP-based agent agents described herein, and variants thereof). The capsule includes a plurality of enteric-coated AP-based agent-containing pellets. In such embodiments, the formulation comprises about 10.0% by weight AP-based agent (e.g. IAP, or the other AP-based agent agents described herein, and variants thereof); about 38.9% by weight sucrose sphere; about 20.0% by weight hydroxypropylcellulose (HPC); about 0.3% by weight of buffer salt; about 26.3% by weight enteric polymer (e.g., EUDRAGIT L 30 D-55), and about 4.5% by weight HTP-20 (e.g., PLASACRYL HTP 20).

In embodiments, the IAP and the composition comprising one or more CPIs are co-formulated.

In embodiments, the formulation comprising IAP is resistant to compression and therefore suitable for tableting. In embodiments, the formulation comprising one or more CPIs is resistant to compression and therefore suitable for tableting. The IAP can be provided in a powder form that is then tableted, e.g., by physical compression of dried materials.

In embodiments, the formulation includes one or more enzyme co-factors, such as zinc and/or magnesium. In embodiments, the enzyme co-factor zinc is used. In embodiments, the zinc is provided as zinc sulfate heptahydrate. In embodiments, the enzyme co-factor magnesium is used. In embodiments, the magnesium is provided as magnesium sulfate heptahydrate.

In embodiments, the formulation includes a protein stabilizer such as trehalose, sucrose, lactose, mannitol, Tween) 80, and/or polyvinyl alcohol. In embodiments, the stabilizer is arginine. In embodiments, the stabilizer is sucrose. In embodiments, the stabilizer is lactose.

In embodiments, the formulation includes one or more surfactants. The surfactants can be used as solubilizers or emulsifying agents. Illustrative surfactants include, but are not limited to, vitamin E polyethylene glycol succinate, sorbitan monostearate-60/80, polysorbate 20, polysorbate 80, and polyoxyl 40 hydrogenated castor oil.

In embodiments, the IAP and/or composition comprising one or more CPIs of the disclosure is stable and/or active in the GI tract, e.g. in one or more of the mouth, esophagus, stomach, duodenum, small intestine, duodenum, jejunum, ileum, large intestine, colon transversum, colon descendens, colon ascendens, colon sigmoidenum, cecum, and rectum. In a specific embodiment, the IAP and/or composition comprising one or more CPIs is stable in the large intestine, optionally selected from one or more of colon transversum, colon descendens, colon ascendens, colon sigmoidenum and cecum. In a specific embodiment, the IAP and/or composition comprising one or more CPIs is stable in the small intestine, optionally selected from one or more of duodenum, jejunum, and ileum. In embodiments, the IAP and/or composition comprising one or more CPIs is resistant to proteases in the GI tract, including for example, the small intestine. In embodiments, the IAP composition comprising one or more CPIs is substantially active at a pH of about 5.0 or above. For example, the IAP and/or composition comprising one or more CPIs may be substantially active at a pH of about 6.0 to about 12, e.g. about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or about 7.3, or about 7.4, or about 7.5, or about 8.0, or about 8.5, or about 9.0, or about 9.5, or about 10.0, or about 10.5, or about 11.0, or about 11.5, or about 12.0 (including, for example, via formulation, as described herein). In embodiments, stable refers to an enzyme that has a long enough half-life and maintains sufficient activity for therapeutic effectiveness.

In embodiments, the IAP and/composition comprising one or more CPIs of the disclosure is stable in chyme, gastric juice, and/or bile salts. In order to assess IAP and/or composition comprising one or more CPIs stability in chyme, samples of IAPs and composition comprising one or more CPIs are incubated in human chyme at 37° C. Stability is then evaluated by assessing aliquots withdrawn from the incubated samples at 0, 0.5, 1, 2, 3, 4, 5, and 6 hours for AP activity using a para-nitrophenyl phosphate (pNPP) AP substrate. Different chyme specimens can be used for evaluation of stability, including mixed chyme samples. Chyme samples are characterized for pH, liquid content, and protease activity.

In embodiments, the IAP described herein includes derivatives that are modified, i.e., by the covalent attachment of any type of molecule to the alkaline phosphatase such that covalent attachment does not prevent the activity of the enzyme. For example, but not by way of limitation, derivatives include alkaline phosphatases that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out, including, but not limited to specific chemical cleavage, acetylation, formylation, metabolic synthesis of tunicamycin, etc. Additionally, the derivative can contain one or more non-classical amino acids. In embodiments, the IAP is glycosylated to ensure proper protein folding.

In embodiments, formulations and unit dose forms herein, as well as those used in methods herein, include co-formulation, co-administration, and/or formulation components of that described in U.S. Pat. No. 10,987,410, and Published U.S. Patent Applications US20210030686 and US20220323367, (e.g., alkaline phosphatase formulations and uses thereof), each of which is incorporated herein in their entirety.

### Pharmaceutically Acceptable Salts

The IAP and/or composition comprising one or more CPIs described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

The term “pharmaceutically acceptable salt” also refers to a salt of the alkaline phosphatases having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In embodiments, the compositions described herein are in the form of pharmaceutically acceptable salts. In embodiments, the formulation comprises 5%, about 10%, about 15%, about 20%, about 25%, about 30%, about 31%, about 32%, about 33%, about 34%, about 35%, about 36%, about 37%, about 38%, about 39%, about 40%, about 41%, about 42%, about 43%, about 43%, about 44%, about 45%, about 46%, about 47%, about 48%, about 49%, or about 50% by weight pharmaceutically acceptable salts.

### Pharmaceutical Excipients

Further, any IAP and/or composition comprising one or more CPIs described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, cellulose, hypromellose, lactose, sucrose, trehalose, malt, rice, flour, chalk, silica gel, sodium stearate, glycerol monostearate, talc, sodium chloride, dried skim milk, glycerol, propylene, glycol, povidone, crosspovidone, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

A suitable pharmaceutical excipient for the purposes of tableting can be Ludipress (Lactose, povidone, crospovidone; CAS-No.: 5989-81-1+9003-39-8).

Where necessary, the IAP and/or composition comprising one or more CPIs and/or pharmaceutical compositions (and/or additional therapeutic agents) can include a solubilizing agent. Also, the agents can be delivered with a suitable vehicle or delivery device. Combination therapies outlined herein can be co-delivered in a single delivery vehicle or delivery device.

In one embodiment, the IAP and/or composition comprising one or more CPIs (and/or additional therapeutic agents) described herein are formulated as compositions adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide a sustained action over an extended period of time. Selectively permeable membranes surrounding an osmotically active agent driving any IAP (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include excipients such as mannitol, lactose, starch, magnesium stearate, sodium saccharin, cellulose, ethacrylic acid and derivative polymers thereof, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In embodiments, the IAP and/or composition comprising one or more CPIs (and/or additional therapeutic agent) are formulated as solid dosage forms such as tablets, dispersible powders, granules, and capsules. In one embodiment, the IAP and/or composition comprising one or more CPIs (and/or additional therapeutic agent) are formulated as a capsule. In embodiments, the IAP and/or composition comprising one or more CPIs (and/or additional therapeutic agent) are formulated as a tablet. In yet embodiments, the IAP and/or composition comprising one or more CPIs (and/or additional therapeutic agent) are formulated as a soft-gel capsule. In embodiments, the IAP and/or composition comprising one or more CPIs (and/or additional therapeutic agent) are formulated as a gelatin capsule.

In embodiments, the formulations of the IAP and/or composition comprising one or more CPIs may additionally comprise a pharmaceutically acceptable carrier or excipient. As one skilled in the art will recognize, the formulations can be in any suitable form appropriate for the desired use and route of administration.

In some dosage forms, the agents described herein are mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients may have two or more functions in the oral dosage form. In the case of an oral dosage form, for example, a capsule or a tablet, the dosage form may also comprise buffering agents.

### Surface Active Agents

Any formulation described herein can additionally include a surface active agent. Surface active agents suitable for use in the present disclosure include, but are not limited to, any pharmaceutically acceptable, non-toxic surfactant. Classes of surfactants suitable for use in the compositions of the disclosure include, but are not limited to polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In embodiments, compositions of the disclosure may comprise one or more surfactants including, but not limited to, sodium lauryl sulfate, polysorbate 20, polysorbate 40, polysorbate 60, polysorbate 80, and triethyl citrate.

The formulation can also contain pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

The formulation can also include one or more application solvents. Some of the more common solvents that can be used to apply, for example, a delayed-release coating composition include isopropyl alcohol, acetone, methylene chloride and the like.

The formulation can also include one or more alkaline materials. Alkaline material suitable for use in compositions of the disclosure include, but are not limited to, sodium, potassium, calcium, magnesium and aluminum salts of acids such as phosphoric acid, carbonic acid, citric acid and other aluminum/magnesium compounds. In addition, the alkaline material may be selected from antacid materials such as aluminum hydroxides, calcium hydroxides, magnesium hydroxides and magnesium oxide.

In embodiments, the formulation can additionally include magnesium and/or zinc. Without wishing to be bound by theory, the inclusion of magnesium and/or zinc in the formulation promotes protein folding (e.g., dimer formation) and bioactivity of the IAP. In embodiments, the formulation can include magnesium at a concentration of from about 1 μM to greater than 5 mM (e.g., from about 1 μM to more than 5 mM), inclusive of all ranges and values therebetween. In embodiments, the magnesium is present in the formulation at 1.0 mM. In embodiments, the formulation can include zinc at a concentration of about 1 μM to greater than 1 mM (e.g., from about 1 μM to more than 1 mM), inclusive of all ranges and values therebetween. In embodiments, the zinc is present in the formulation at 0.1 mM. In embodiments, the formulation of the present disclosure is substantially free of metal chelators.

In embodiments, the pH of the formulation ensures that the IAP is properly folded (e.g., dimer formation) and is bioactive. In embodiments, the formulation is maintained at a pH such that the amino acids which coordinate the binding of magnesium and/or zinc within the AP-based agent are not protonated. Protonation of such coordinating amino acids may lead to loss of metal ions and bioactivity and dimer disassociation. In embodiments, the pH of the formulation is greater than about 6, about 6.5, about 7, about 7.5, about 8, about 8.5, about 9, about 9.5, about 10, about 10.5, about 11, about 11.5, or about 12.

Besides inert diluents, the oral compositions can also include adjuvants such as sweetening, flavoring, and perfuming agents.

### Delivery

Various methods may be used to formulate and/or deliver the agents described herein to a location of interest. For example, the IAP and/or composition comprising one or more CPIs (and/or additional therapeutic agents) described herein may be formulated for delivery to the GI tract. The GI tract includes organs of the digestive system such as mouth, esophagus, stomach, duodenum, small intestine, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the IAP composition comprising one or more CPIs (and/or additional therapeutic agents) described herein may be formulated for delivery to one or more of the stomach, small intestine, large intestine and rectum and includes all subsections thereof (e.g. duodenum, jejunum and ileum, colon transversum, colon descendens, colon ascendens, colon sigmoidenum and cecum). In embodiments, the compositions described herein may be formulated to deliver to the gut. In embodiments, the compositions described herein may be formulated to deliver to the upper or lower GI tract. In embodiments, the IAP and/or composition comprising one or more CPIs (and/or additional therapeutic agents) may be administered to a subject, by, for example, directly or indirectly contacting the mucosal tissues of the GI tract. In embodiments, the administration of the IAP and/or composition comprising one or more CPIs (and/or additional therapeutic agents) is into the GI tract via, for example, oral delivery, nasogastral tube, intestinal intubation (e.g. an enteral tube or feeding tube such as, for example, a jejunal tube or gastro-jejunal tube, etc.), direct infusion (e.g., duodenal infusion), endoscopy, colonoscopy, sigmoidoscopy or enema.

For example, in embodiments, the present disclosure provides modified release formulations comprising at least one IAP (and/or additional therapeutic agents), wherein the formulation releases a substantial amount of the IAP (and/or additional therapeutic agents) into one or more regions of the GI tract. For example, the formulation may release at least about 60% of the AP-based agent after the stomach and into one or more regions of the GI tract.

In embodiments, the IAP is formulated to be substantially released in the GI tract. In embodiments, the IAP is formulated to be substantially released in the small intestine. In embodiments, the IAP is formulated to be substantially released in the large intestine. In embodiments, the IAP is formulated to not be substantially released systemically.

In embodiments, the modified-release formulation of the present disclosure releases at least 60% of the IAP (or additional therapeutic agents) after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the IAP (or additional therapeutic agents) in the intestines.

In embodiments, the modified-release formulation of the present disclosure releases at least 60% of the IAP (or additional therapeutic agents) in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the IAP (or additional therapeutic agents) in the small intestine (e.g., one or more of duodenum, jejunum, ileum, and ileocecal junction).

In embodiments, the modified-release formulation of the present disclosure releases at least 60% of the IAP (or additional therapeutic agents) in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the IAP (or additional therapeutic agents) in the large intestine (e.g., one or more of cecum, ascending, transverse, descending or sigmoid portions of the colon, and rectum).

In embodiments, the modified-release formulation does not substantially release the IAP (or additional therapeutic agents) in the stomach.

In certain embodiments, the modified-release formulation releases the IAP and/or composition comprising one or more CPIs (or additional therapeutic agents) above a specific pH. For example, in embodiments, the modified-release formulation is substantially stable in an acidic environment and substantially unstable (e.g., dissolves rapidly or is physically unstable) in a near neutral to alkaline environment. In embodiments, stability is indicative of not substantially releasing while instability is indicative of substantially releasing. For example, in embodiments, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In embodiments, the present formulations are stable in lower pH areas and therefore do not substantially release in, for example, the stomach. In embodiments, the modified-release formulation is substantially stable at a pH of about 1 to about 5 or lower and substantially unstable at pH values that are greater. In these embodiments, the modified-release formulation does not substantially release in the stomach. In these embodiments, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In embodiments, modified-release formulation is substantially stable at a pH of about 4 to about 7 or lower and consequentially is substantially unstable at pH values that are greater and therefore is not substantially released in the stomach and/or proximal small intestine (e.g. one or more of the duodenum, jejunum). In these embodiments, the modified-release formulation substantially releases in the distal small intestine or large intestine (e.g. one or more of the cecum, ascending colon, transverse colon, descending colon, and sigmoid colon). In embodiments, the pH values recited herein may be adjusted as known in the art to account for the state of the subject, e.g. whether in a fasting or postprandial state.

In embodiments, the modified-release formulation is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In embodiments, the modified-release formulation is stable in gastric fluid or stable in acidic environments. These modified-release formulations release about 30% or less by weight of the alkaline phosphatase and/or additional therapeutic agent in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of the of the disclosure may release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the alkaline phosphatase and/or additional therapeutic agent in the modified-release formulation in gastric fluid with a pH of 4-5, or less or simulated gastric fluid with a pH of 4-5 or less, in about 15, or about 30, or about 45, or about 60, or about 90 minutes. Modified-release formulations of the disclosure may release about 1%, about 2%, about 3%, about 4%, about 5%, about 6%, about 7%, about 8%, about 9%, or about 10% by weight of the total alkaline phosphatase and/or additional therapeutic agent in the modified-release formulation in gastric fluid with a pH of 5 or less, or simulated gastric fluid with a pH of 5 or less, in about 15, or about 30, or about 45, or about 60, or about 90 minutes.

In embodiments, the modified-release formulation is unstable in intestinal fluid. These modified-release formulations release about 70% or more by weight of the alkaline phosphatase and/or additional therapeutic agent in the modified-release formulation in intestinal fluid or simulated intestinal fluid in about 15, or about 30, or about 45, or about 60, or about 90 minutes. In embodiments, the modified-release formulation is unstable in near neutral to alkaline environments. These modified-release formulations release about 70% or more by weight of the alkaline phosphatase and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments may release 70% or more by weight of alkaline phosphatase and/or additional therapeutic agent in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In embodiments, the modified-release formulation of the disclosure is substantially stable in chyme. For example, there is, in embodiments, a loss of less than about 50% or about 40%, or about 30%, or about 20%, or about 10% of AP-based agent activity in about 10, or 9, or 8, or 7, or 6, or 5, or 4, or 3, or 2, or 1 hour from administration.

In embodiments, the modified-release formulations of the present disclosure are designed for immediate release (e.g. upon ingestion). In embodiments, the modified-release formulations may have sustained-release profiles, i.e. slow release of the active ingredient(s) in the body (e.g., GI tract) over an extended period of time. In embodiments, the modified-release formulations may have a delayed-release profile, i.e. not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric-coated to delay release of the active ingredient(s) until it reaches the small intestine or large intestine.

### Enteric Coating

In embodiments, the formulations of the present disclosure (e.g. IAP as a powder or tablet) are coated to provide protection of the active agent in the GI tract, including the stomach. For example, in embodiments, the present formulations can be encapsulated in an enterically-coated capsule. Additionally, in embodiments, the formulations (e.g. IAP as a powder or tablet) itself is coated with one or more coatings, e.g. one or more modified-release coatings as described herein (e.g. after a step of granulating the powder). Further, in embodiments, the present powder formulations (e.g. IAP as a powder) can be compressed into a tablet that is enterically coated.

In embodiments, the modified-release formulation of the present disclosure may utilize one or more modified-release coatings such as delayed-release coatings to provide for effective, delayed yet substantial delivery of the alkaline phosphatase to the GI tract together with, optionally, additional therapeutic agents.

In embodiments, the modified-release formulation of the present disclosure may utilize one or more modified-release coatings such as delayed-release coatings to provide for effective, delayed yet substantial delivery of the IAP to the intestines together with, optionally, other additional therapeutic agents.

In one embodiment, the delayed-release coating includes an enteric agent that is substantially stable in acidic environments and substantially unstable in near neutral to alkaline environments. In embodiments, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The polymers are described in international pharmacopeias such as Ph. Eur., USP/NF, DMF, and JPE. The EUDRAGIT®-type polymers include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. Similar polymers include various KOLLICOAT (e.g., polyvinyl alcohol, PEG, and colloidal anhydrous silica) (e.g., Kollicoat® MAE 30 DP or Kollicoat® MAE 100 P) and EUDRAGIT (e.g., polymethacrylate-based copolymers) polymers and formulations. In embodiments, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In embodiments, the enteric agent may be a combination of the foregoing solutions or dispersions. In embodiments, the delayed-release coating includes the enteric agent EUDRAGIT® L 100.

By way of non-limiting example, there are various EUDRAGIT formulations that dissolve at rising pH, with formulations that dissolve at pH >5.5 (EUDRAGIT L30 D-550), pH >6.0 (EUDRAGIT L12, 5), and pH >7.0 (EUDRAGIT FS 30D). Since the ileum has the highest pH in the small intestine, ranging from 7.3 to 7.8, the use of EUDRAGIT FS 30D as an enteric agent, may delay dissolution until the ileum thereby localizing the release of the AP-based agent to the ileum. However, the jejunum has a pH that can range from 6.6 to 7.4, therefore, various EUDRAGIT formulations can be used to target release to this segment of the intestine. The different types of EUDRAGIT can be combined with each other, or multiple different types of EUDRAGIT coatings can be combined to fine tune the dissolution profile to achieve targeted delivery to achieve optimal function. For example, EUDRAGIT L100, EUDRAGIT S100, and triethyl citrate may be mixed together at a ratio of, for example, about 72.7/18.2/9.1, to form a coating that substantially releases at a pH of greater than about 6.2. In another example, EUDRAGIT L100, EUDRAGIT S100, and triethyl citrate may be mixed together at a ratio of, for example, about 30/60.9/9, to form a coating that substantially releases at a pH of greater than about 6.7. In a further example, DuoCoat™ (Kuecept, Ltd.) may be used that uses two coatings of enteric polymers (like EUDRAGIT), an outer layer, and an inner layer of partially neutralized enteric polymer and a buffer agent. The DuoCoat™ technology allows more rapid release of the therapeutic agent initiated at the targeted pH compared to a single coating of the enteric polymer (Liu et al., 2010, European J. Pharmaceutics and Biopharmaceuticals 47:311, the entire contents of all of which are incorporated herein by reference). Release was demonstrated to be targeted to the ileum and/or ileoceacal junction in 10 healthy volunteers (Varum et al., 2013, European J. Pharmaceutics and Biopharmaceuticals 84:573, the entire contents of all of which are incorporated herein by reference).

In certain embodiments, one or more coating system additives are used with the enteric agent. For example, one or more PLASACRYL (e.g., emulsions containing varying ratios of anti-tacking mono and di-glycerides (glyceryl monostearate (GMS)), plasticizers (triethyl citrate (TEC)), and/or stabilizers (polymethacrylate-based copolymer)) additives may be used as an anti-tacking agent coating additive, plasticizing additive, and/or stabilizing additive. Illustrative PlasACRYL™ additives include, but are not limited to PlasACRYL™MHTP20 and PlasACRYL™T20.

In embodiments, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers useful in the present disclosure include polyvinyl esters, polyvinyl acetals, polyacrylic acid esters, butadiene styrene copolymers, and the like. In one embodiment, colonic delivery is achieved by use of a slowly-eroding wax plug (e.g., various PEGS, including for example, PEG6000) or pectin. In embodiments, the present disclosure contemplates the use of a delayed-release coating that degrade as a function of time which comprises a swell layer comprising croscarmellos sodium and hydroxyproplycellulose. In such embodiment, the formulation may further include an osmotic rupture coating that comprises ethylcellulose such as ethylcellulose dispersions.

Alternatively, the stability of the modified-release formulation can be enzyme-dependent. Delayed-release coatings that are enzyme dependent will be substantially stable in fluid that does not contain a particular enzyme and substantially unstable in fluid containing the enzyme. The delayed-release coating will essentially disintegrate or dissolve in fluid containing the appropriate enzyme. Enzyme-dependent control can be brought about, for example, by using materials which release the active ingredient only on exposure to enzymes in the intestine, such as galactomannans. Also, the stability of the modified-release formulation can be dependent on enzyme stability in the presence of a microbial enzyme present in the gut flora. For example, in embodiments, the delayed-release coating may be degraded by a microbial enzyme present in the gut flora. In embodiments, the delayed-release coating may be degraded by bacteria present in the small intestine. In embodiments, the delayed-release coating may be degraded by bacteria present in the large intestine.

In embodiments, the modified release formulation is designed for release in the colon. Various colon-specific delivery approaches may be utilized. For example, the modified release formulation may be formulated using a colon-specific drug delivery system (CODES) as described for example, in Li et al., AAPS PharmSciTech (2002), 3 (4): 1-9, the entire contents of which are incorporated herein by reference. Drug release in such a system is triggered by colonic microflora coupled with pH-sensitive polymer coatings. For example, the formulation may be designed as a core tablet with three layers of polymer. The first coating is an acid-soluble polymer (e.g., EUDRAGIT E), the outer coating is enteric, along with a hydroxypropyl methylcellulose barrier layer interposed in between. In embodiments, colon delivery may be achieved by formulating the alkaline phosphatase (and/or additional therapeutic agent) with specific polymers that degrade in the colon such as, for example, pectin. The pectin may be further gelled or crosslinked with a cation such as a zinc cation. In embodiments, the formulation is in the form of ionically crosslinked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDS-CT).

Formulations for colon specific delivery of the IAP and/or composition comprising one or more CPIs (and/or additional therapeutic agents), as described herein, may be evaluated using, for example, in vitro dissolution tests. For example, parallel dissolution studies in different buffers may be undertaken to characterize the behavior of the formulations at different PH levels. Alternatively, in vitro enzymatic tests may be carried out. For example, the formulations may be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In embodiments, in vivo evaluations may be carried out using animal models such as dogs, guinea pigs, rats, and pigs. Further, clinical evaluation of colon specific drug delivery formulations may be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon may be monitored by colonoscopy and intubation.

In embodiments, the present formulations provide for substantial uniform dissolution of the IAP (and/or additional therapeutic agent) in the area of release in the GI tract. In embodiments, the present formulation minimizes patchy or heterogeneous release of the AP-based agent.

In embodiments, the present disclosure provides for modified-release formulations that release multiple doses of the AP-based agent, at different locations along the intestines, at different times, and/or at different pH. In embodiments, the modified-release formulation comprises a first dose of the IAP and a second dose of the IAP, wherein the first dose and the second dose are released at different locations along the intestines, at different times, and/or at different pH. For example, the first dose is released at the duodenum, and the second dose is released at the ileum. In another example, the first dose is released at the jejunum, and the second dose is released at the ileum. In embodiments, the first dose is released at a location along the small intestine (e.g., the duodenum), while the second dose is released along the large intestine (e.g., the ascending colon). In embodiments, the modified-release formulation may release at least one dose, at least two doses, at least three doses, at least four doses, at least five doses, at least six doses, at least seven doses, or at least eight doses of the AP-based agent at different locations along the intestines, at different times, and/or at different pH.

In embodiments, the formulations of the present disclosure take the form of those as described in one or more of U.S. Pat. Nos. 8,535,713 and 8,911,777 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In embodiments, the formulations of the present disclosure take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

In embodiments, the process of formulating the IAP is sufficiently gentle such that the tertiary structure of the IAP (e.g., dimeric structure) is substantially intact. In embodiments, the process of formulating the IAP includes a step of refolding the IAP. In such embodiments, the step of refolding the IAP may include the addition of magnesium and/or cyclodextrin.

In embodiments, the modified-release formulation is a modified-release powder formulation.

In embodiments, the modified-release formulation including IAPs described herein, and variants thereof, and/or additional therapeutic agents is administered orally.

Suitable dosage forms for oral use include, for example, solid dosage forms such as tablets, capsules, powders, and granules. In embodiments, the modified-release formulation is in the form of powders. In embodiments, the powdered formulations of the present disclosure can be added to food (e.g. juices, strained and/or pureed foods (e.g. fruits, vegetables), sauces, infant formulas, milk, etc.). In embodiments, the modified-release formulation is packaged in the form of a sachet. In embodiments, the modified-release formulation is in the form of tablets. In embodiments, the modified-release formulation is in the form of tablets comprising powders. In embodiments, the modified-release formulation is in the form of capsules. In embodiments, the modified-release formulation is in the form of capsules comprising powders.

In embodiments, the modified-release formulation of the disclosure is in the form of powders. In embodiments, the powders are formed by spray drying and/or by spray-dried dispersion (SDD) technology. In embodiments, the powders comprising AP-based agents are formed by dissolving AP-based agents and polymers in a solvent and then spray-drying the solution. The resulting powder comprises the AP-based agents dispersed within a solid polymeric matrix.

Various types of polymers may be used for the modified-release formulation of the disclosure. In embodiments, the polymer is an enteric polymer that is substantially stable in acidic environments and substantially unstable in near neutral to alkaline environments. In embodiments, the enteric polymer is substantially stable in gastric fluid.

Illustrative polymers include, but are not limited to, copovidone, polyvinyl caprolactam-polyvinyl acetate-polyethyleneglycol copolymer, poly(vinylpyrrolidinone) (PVP), hydroxypropylmethylcellulose or hypromellose (HPMC), hypromellose phthalate (HPMCP), hydroxypropylmethylcellulose or hypromellose acetate succinate (HPMCAS), methacrylate/methacrylic acid copolymer, and mixtures thereof. In embodiments, the polymer is HPMCAS. In embodiments, the poymer is HPMCAS LF, LG, MF, MG, HF, or HG. In embodiments, the polymer is HPMCAS-LF.

In embodiments, the modified-release formulation further comprises a single layer enteric coating having about 20-40% enteric polymer weight gain, optionally having about 26.3% enteric polymer weight gain. In embodiments, the capsule comprises gelatin or hydroxypropyl methylcellulose.

### Buffers

In embodiments, various types of solvents/buffers are used for preparation of the powders of the disclosure. In embodiments, the solvents/buffers are organic solvents/buffers. Illustrative solvents/buffers that may be used to dissolve the AP-based agent and polymer prior to spray-drying include, but are not limited to, ethanol, methanol, acetone, IPA, tetrahydrafuran, dichloromethane, and mixtures thereof. In embodiments, the solvent used is water such as distilled DI water. In embodiments, the buffer used is monosodium phosphate monohydrate.

In embodiments, various buffers, such as Good's Buffers (e.g., MES, ADA, PIPES, ACES, MOPSO, Chloramine chloride, MOPS, BES, TES, HEPES, DIPSO, TAPSO, Acetamidoglycine, POPSO, HEPPSO, HEPPS, Tricine, Tris, Glycinamide, Glycylglycine, Bicine, and/or TAPS) are used, as well as salts of these buffers. In embodiments, buffers include amino acid buffers, such as in non-limiting examples, histidine, arginine, and/or cysteine. In embodiments, buffers include phosphate-buffered saline (PBS). In embodiments, various salts are used, such as in non-limiting examples, potassium chloride, sodium chloride, calcium chloride, magnesium chloride, sodium sulfate, calcium sulfate, and/or magnesium sulfate. In embodiments, the buffer and/or salt includes a combination of any one of potassium chloride, phosphoric acid, calcium chloride, magnesium sulfate, potassium phosphate (monobasic and/or dibasic), and/or sodium phosphate (monobasic and/or dibasic). In embodiments, the buffer used is monosodium phosphate monohydrate. In embodiments, the buffer used includes arginine and phosphate.

In embodiments, enzyme co-factors including zinc and magnesium are used. In embodiments, the enzyme co-factor zinc is used. In embodiments, the zinc is provided as zinc sulfate heptahydrate. In embodiments, the enzyme co-factor magnesium is used. In embodiments, the magnesium is provided as magnesium sulfate heptahydrate.

In embodiments, the formulation includes a protein stabilizer such as trehalose, sucrose, lactose, mannitol, Tween 80, or polyvinyl alcohol. In embodiments, the stabilizer is sucrose. In embodiments, the stabilizer is lactose.

In embodiments, surfactants may be included for the preparation of the powders of the disclosure. The surfactants may be used as solubilizers or emulsifying agents. Illustrative surfactants include, but are not limited to, vitamin E polyethylene glycol succinate, sorbitan monostearate-60/80, polysorbate 20, polysorbate 80, and polyoxyl 40 hydrogenated castor oil.

In embodiments, the powders comprising AP-based agents becomes a gel. In embodiments, the powders comprising an AP-based agent becomes a gel in the intestines. In embodiments, the AP-based agent is released from the gel into one or more regions of the intestines. In embodiments, at pH values greater than about 5 (e.g. about 5, or 6, or 7, or 8, or 9) the gel transforms back into the solution phase and releases the AP enzyme. In embodiments, the gel is used to control the release of the AP-based agent in the intestines. In embodiments, the AP-based agent is released from the gel into one or more of the group consisting of the small intestine, duodenum, jejunum, ileum, large intestine, colon transversum, colon descendens, colon ascendens, colon sigmoidenum, cecum, and rectum.

In embodiments, the formulation of the present disclosure is in the form of powders comprising the AP-based agent dispersed within a solid polymeric matrix. In embodiments, the powders are formed by dissolving AP-based agent and polymers in a solvent to form a solution that is subsequently spray-dried. In embodiments, the solution for spray-drying comprises about 0.1-1% by weight of AP-based agent. For example, the AP-based agent may be present about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1.0% by weight. In embodiments, the solution comprises about 1-10% by weight a polymer (e.g., HPMCAS-LF). For example, the polymer may be present at about 1%, about 2%, about 3%, about 4%, about 5%, about 6%, about 7%, about 8%, about 9%, or about 10% by weight. In embodiments, the solution comprises about 0.05-0.5% by weight buffer (e.g., monosodium phosphate monohydrate). For example, the buffer may be present at about 0.05%, about 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19%, about 0.20%, about 0.25%, about 0.30%, about 0.35%, about 0.40%, about 0.45%, or about 0.50% by weight. In embodiments, the solution comprises about 0.001-0.01% by weight zinc (e.g., zinc sulfate heptahhydrate). For example, the zinc may be present at about 0.001%, about 0.002%, about 0.003%, about 0.004%, about 0.005%, about 0.006%, about 0.007%, about 0.008%, about 0.009%, or about 0.01% by weight. In embodiments, the solution comprises about 0.01-0.1% by weight magnesium (e.g., magnesium sulfate heptahhydrate). For example, the magnesium may be present at about 0.01%, about 0.02%, about 0.03%, about 0.04%, about 0.05%, about 0.06%, about 0.07%, about 0.08%, about 0.09%, or about 0.1% by weight. In embodiments, the solution comprises about 0.1-1% by weight a protein stabilizer (e.g., trehalose). For example, the protein stabilizer may be present at about 0.1%, about 0.2%, about 0.3%, about 0.4%, about 0.5%, about 0.6%, about 0.7%, about 0.8%, about 0.9%, or about 1% by weight. In embodiments, the solution comprises about 90-99.9% by weight solvent (e.g., water). For example, the solvent may be present at about 90%, about 91%, about 92%, about 93%, about 94%, about 95%, about 96%, about 97%, about 98%, or about 99% by weight.

In embodiments, the IAP is formulated in a modified release formulation comprising at least one modified release pellet. In embodiments, the modified-release formulation of the disclosure is in the form of tablets or capsules. In embodiments, the modified-release formulation is in the form of tablets or capsules comprising the powders of the disclosure. A variety of approaches for generating tablets or capsules may be utilized to include powders of the disclosure. In embodiments, tablets of the disclosure are generated by granulation such as dry granulation. In such embodiments, the powders are pre-compressed and the resulting tablet or slug is milled to yield granules. Alternatively, the powders are pre-compressed with pressure rolls to yield granules. In yet embodiments, the powders are encapsulated into capsules. In embodiments, the capsule is a gelatin capsule, such as a hard gelatin capsule. In embodiments, the capsule is a hydroxypropyl methylcellulose (HPMC) capsule.

In embodiments, the tablets or capsules comprise a delayed-release coating that includes an enteric agent that is substantially stable in acidic environments and substantially unstable in near neutral to alkaline environments. In embodiments, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The polymers are described in international pharmacopeias such as Ph. Eur., USP/NF, DMF, and JPE. The EUDRAGIT®-type polymers include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. Similar polymers include Kollicoat® MAE 30 DP and Kollicoat® MAE 100 P. In embodiments, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In embodiments, the enteric agent may be a combination of the foregoing solutions or dispersions. In embodiments, the delayed-release coating includes the enteric agent EUDRAGIT® L 100. In embodiments, the tablet or capsule is coated with the enteric agent at a coating weight of about 1-20% such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% coating weight.

### Administration and Dosages

It will be appreciated that the actual dose of the IAP and/or composition comprising one or more CPIs (and/or additional therapeutic agents) to be administered according to the present disclosure will vary according to the particular compound, the particular dosage form, and the mode of administration. Many factors that may modify the action of the AP-based agent (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

Individual doses of the IAP and/or composition comprising one or more CPIs (and/or additional therapeutic agents) can be administered in unit dosage forms (e.g., tablets or capsules) containing, for example, from about 0.01 mg to about 1,000 mg, about 0.01 mg to about 900 mg, about 0.01 mg to about 800 mg, about 0.01 mg to about 700 mg, about 0.01 mg to about 600 mg, about 0.01 mg to about 500 mg, about 0.01 mg to about 400 mg, about 0.01 mg to about 300 mg, about 0.01 mg to about 200 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, or from about 0.1 mg to about 1 mg active ingredient per unit dosage for. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg of the AP-based agent, inclusive of all values and ranges therebetween.

In one embodiment, the IAP and/or composition comprising one or more CPIs (and/or additional therapeutic agents) is administered at an amount of from about 0.01 mg to about 1,000 mg daily, about 0.01 mg to about 900 mg daily, about 0.01 mg to about 800 mg daily, about 0.01 mg to about 700 mg daily, about 0.01 mg to about 600 mg daily, about 0.01 mg to about 500 mg daily, about 0.01 mg to about 400 mg daily, about 0.01 mg to about 300 mg daily, about 0.01 mg to about 200 mg daily, about 0.01 mg to about 100 mg daily, an amount of from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In embodiments, the IAP is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 200 mg, about 300 mg, about 400 mg, about 500 mg, about 600 mg, about 700 mg, about 800 mg, about 900 mg, or about 1,000 mg, inclusive of all values and ranges therebetween.

In embodiments, a suitable dosage of the IAP and/composition comprising one or more CPIs (and/or additional therapeutic agents) is in a range of about 0.01 mg/kg to about 100 mg/kg of body weight of the subject, about 0.01 mg/kg to about 90 mg/kg of body weight of the subject, about 0.01 mg/kg to about 80 mg/kg of body weight of the subject, about 0.01 mg/kg to about 70 mg/kg of body weight of the subject, about 0.01 mg/kg to about 60 mg/kg of body weight of the subject, about 0.01 mg/kg to about 50 mg/kg of body weight of the subject, about 0.01 mg/kg to about 40 mg/kg of body weight of the subject, about 0.01 mg/kg to about 30 mg/kg of body weight of the subject, about 0.01 mg/kg to about 20 mg/kg of body weight of the subject, about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In embodiments, a suitable dosage of the AP-based agent is in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain embodiments of the disclosure, the IAP and/or composition comprising one or more CPIs (and/or additional therapeutic agents) may be administered, for example, more than once daily (e.g., about two, about three, about four, about five, about six, about seven, about eight, about nine, or about ten times per day), about once per day, about every other day, about every third day, about once a week, about once every two weeks, about once every month, about once every two months, about once every three months, about once every six months, or about once every year.

In embodiments, the IAP is administered in multiple doses. In embodiments, the IAP is administered in multiple oral doses. In embodiments, the dosing of IAP is set as a function of the amount of IAP recovered in a biological sample of the subject to be treated (e.g., as present in stool, blood, etc.). In embodiments, the dosing of IAP is altered as a function of the severity and/or presence of one or more symptoms.

### Methods of Treatment

In embodiments, IAP and/or a composition comprising one or more CPIs (and/or additional therapeutic agents), including but not limited to one or more agents that modulate one or more of PD-1, PD-L1, PD-L2, ICOS, ICOSL, and CTLA-4, of the present disclosure are co-administered. The co-administration can occur simultaneously or sequentially. In embodiments, the IAP is administered before the CPI.

In embodiments, the present disclosure provides methods of treating or preventing CPI-mediated GI side effects, such as, but not limited to, diarrhea and/or colitis. Colitis can refer to inflammation of the inner lining of the colon. In embodiments, the colitis is associated with abdominal pain, cramping, diarrhea (with or without blood in the stool) and combinations thereof. For example, a patient experiencing colitis may find blood in the bowel movement and experience the constant urge to have a bowel movement (e.g., tenesmus), as well as persistent abdominal pain, and fever, chills, and/or other signs of infection and inflammation.

Accordingly, in embodiments, the methods of the present disclosure provide for preventing and/or treating colitis as a CPI-mediated GI side effect by administering IAP and/or a composition comprising an CPI selected from an agent that modulates one or more of PD-1, PD-L1, PD-L2, ICOS, ICOSL, and CTLA-4. In embodiments, the patient is a cancer patient and may be undergoing therapy with an immune checkpoint inhibitor immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

In embodiments, the methods and uses of the present disclosure include use of IAP and/or a composition comprising one or more CPIs (and/or additional therapeutic agents) as an adjuvant to any of these initial and/or adjunctive therapies (including co-administration or sequential administration). In embodiments, the methods and uses of the present disclosure include administration of the IAP and/or a composition comprising one or more CPIs (and/or additional therapeutic agents), including but not limited to an agent that modulates one or more of PD-1, PD-L1, PD-L2, ICOS, ICOSL, and CTLA-4, described herein to a subject undergoing initial and/or adjunctive therapies.

In embodiments, the IAP is a recombinant bIAP II with a stop codon and no leader sequence, as annotated in SEQ ID NO: 11. In embodiments, the IAP is produced in a mammalian cell line, for example a non-bovine cell line such as CHO cells. In embodiments, the IAP is glycosylated on one or more residues. In embodiments, the IAP is terminally sialylated on one or my glycosylation structures. In embodiments, the IAP possess structure/function characteristics that allow effective dosing at lower doses than would be required by other APs.

In embodiments, IAP of the present disclosure is co-administered with one or more additional therapeutic agents, e.g., as described herein. The co-administration can occur simultaneously or sequentially. In embodiments, the IAP of the present disclosure is capable of being administered with or without additional agents. In embodiments, the methods and uses of the present disclosure include use of IAP as an adjuvant to any of these initial and/or adjunctive therapies (including co-administration or sequential administration). In embodiments, the methods and uses of the present disclosure include administration of the IAP described herein to a subject undergoing initial and/or adjunctive therapies.

In embodiments, methods include administering an IAP that is formulated to be substantially released in the GI tract. In embodiments, methods include administering an IAP that is formulated to be substantially released in the small intestine. In embodiments, methods include administering an IAP that is formulated to be substantially released in the large intestine. In embodiments, methods include administering an IAP that is formulated to not be substantially released systemically. In embodiments, IAP formulated to not be substantially released systemically refers to no detectable IAP found in biological samples such as blood, serum, etc., outside of stool and intestinal lumen samples.

In embodiments, the method includes administrating a recombinant IAP having an amino sequence having about or at least about 90%, about or at least about 95%, about or at least about 96%, about or at least about 97%, about or at least about 98%, or about or at least about 99% or more sequence identity to any one of SEQ ID NOs: 1-14.

In embodiments, the formulation of the present invention is in the form of a capsule (e.g., a hard gelatin or HPMC capsule) comprising about 15 mg of the AP-based agent (e.g. IAP, or the other AP-based agent agents described herein, and variants thereof). The capsule includes a plurality of enteric-coated AP-based agent-containing pellets. In such embodiments, the formulation comprises about 10.0% by weight AP-based agent (e.g. IAP, or the other AP-based agent agents described herein, and variants thereof); about 38.9% by weight sucrose sphere; about 20.0% by weight hydroxypropylcellulose (HPC); about 0.3% by weight of buffer salt; about 26.3% by weight enteric polymer (e.g., EUDRAGIT L 30 D-55), and about 4.5% by weight HTP-20 (e.g., PLASACRYL HTP 20).

In embodiments, the formulation of the present invention is in the form of a capsule (e.g., a hard gelatin or HPMC capsule) comprising about 5 mg of the AP-based agent (e.g. IAP, or the other AP-based agent agents described herein, and variants thereof). The capsule includes a plurality of enteric-coated AP-based agent-containing pellets. In such embodiments, the formulation comprises about 10.0% by weight AP-based agent (e.g. IAP, or the other AP-based agent agents described herein, and variants thereof); about 38.9% by weight sucrose sphere; about 20.0% by weight hydroxypropylcellulose (HPC); about 0.3% by weight of buffer salt; about 26.3% by weight enteric polymer (e.g., EUDRAGIT L 30 D-55), and about 4.5% by weight HTP-20 (e.g., PLASACRYL HTP 20).

In embodiments, the one or more pharmaceutically acceptable excipients comprises about or at least about 1-10% glyceryl monostearate (GMS), triethyl citrate (TEC), and/or polymethacrylate-based copolymer. In embodiments, the capsule includes gelatin or hydroxypropyl methylcellulose.

In embodiments, the IAP and/or additional agents are co-formulated, for example with one or more additional agents intended to address a symptom of IAP or a disease or disorder treated by IAP, as described herein.

In embodiments, the terms “patient” and “subject” are used interchangeably. In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish, a zebra finch, a turtle, or an iguana.

In embodiments, methods of the disclosure are useful in treatment a human patient. In embodiments, the human is a pediatric human. In embodiments, the human is an adult human. In embodiments, the human is a geriatric human. In embodiments, the human is a female. In embodiments, the human is a male.

In certain embodiments, the human patient has an age in a range of from less than 1 year, including neonates to about 1 year old, 1 year old to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old, from about 100 years old to about 105 years old, from about 105 years old to about 110 years old, from about 110 years old to about 115 years old, from about 115 years old to about 120 years old, from about 120 years old to about 125 years old, from about 125 years old to about 130 years old, from about 130 years old to about 135 years old, from about 135 years old to about 140 years old, from about 140 years old to about 145 years old, from about 145 years old to about 150 years old.

### Additional Therapeutic Agents and Combination Therapy

Administration of the present compositions and formulations comprising the IAP and/or a composition comprising one or more CPIs, including but not limited to an agent that modulates one or more of PD-1, PD-L1, PD-L2, ICOS, ICOSL, and CTLA-4, may be combined with additional therapeutic agents. Co-administration of the additional therapeutic agent and the present compositions/formulations may be simultaneous or sequential. Further, the present compositions/formulations may comprise an additional therapeutic agent (e.g. via co-formulation). For example, the additional therapeutic agent and the IAP and/or a composition comprising one or more CPIs, including but not limited to an agent that modulates one or more of PD-1, PD-L1, PD-L2, ICOS, ICOSL, and CTLA-4, may be combined into a single formulation. Alternatively, the additional therapeutic agent and the IAP and/or a composition comprising one or more CPIs, including but not limited to an agent that modulates one or more of PD-1, PD-L1, PD-L2, ICOS, ICOSL, and CTLA-4, may be formulated separately.

In one embodiment, the additional therapeutic agent and the IAP and/or a composition comprising one or more CPIs, including but not limited to an agent that modulates one or more of PD-1, PD-L1, PD-L2, ICOS, ICOSL, and CTLA-4, are administered to a subject simultaneously. The term “simultaneously” as used herein, means that the additional therapeutic agent and the IAP and/or a composition comprising one or more CPIs, including but not limited to an agent that modulates one or more of PD-1, PD-L1, PD-L2, ICOS, ICOSL, and CTLA-4 are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the IAP and/or a composition comprising one or more CPIs, including but not limited to an agent that modulates one or more of PD-1, PD-L1, PD-L2, ICOS, ICOSL, and CTLA-4, can be by simultaneous administration of a single formulation (e.g., a formulation comprising the additional therapeutic agent and the IAP and/or a composition comprising one or more CPIs, including but not limited to an agent that modulates one or more of PD-1, PD-L1, PD-L2, ICOS, ICOSL, and CTLA-4) or of separate formulations (e.g., a first formulation including the additional therapeutic agent and a second formulation including the IAP and/or a composition comprising one or more CPIs, including but not limited to an agent that modulates one or more of PD-1, PD-L1, PD-L2, ICOS, ICOSL, and CTLA-4).

In embodiments, the additional therapeutic agent and the IAP and/or a composition comprising one or more CPIs, including but not limited to an agent that modulates one or more of PD-1, PD-L1, PD-L2, ICOS, ICOSL, and CTLA-4, are administered to a subject simultaneously but the release of the additional therapeutic agent and the IAP and/or a composition comprising one or more CPIs, including but not limited to an agent that modulates one or more of PD-1, PD-L1, PD-L2, ICOS, ICOSL, and CTLA-4, from their respective dosage forms (or single unit dosage form if co-formulated) may occur sequentially.

Co-administration does not require the additional therapeutic agent and the IAP and/or a composition comprising one or more CPIs, including but not limited to an agent that modulates one or more of PD-1, PD-L1, PD-L2, ICOS, ICOSL, and CTLA-4, to be administered simultaneously, if the timing of their administration is such that the pharmacological activities of the additional therapeutic agent and the IAP and/or a composition comprising one or more CPIs, including but not limited to an agent that modulates one or more of PD-1, PD-L1, PD-L2, ICOS, ICOSL, and CTLA-4, overlap in time. For example, the additional therapeutic agent and the IAP and/or a composition comprising one or more CPIs, including but not limited to an agent that modulates one or more of PD-1, PD-L1, PD-L2, ICOS, ICOSL, and CTLA-4, can be administered sequentially. The term “sequentially” as used herein means that the additional therapeutic agent and the IAP and/or a composition comprising one or more CPIs, including but not limited to an agent that modulates one or more of PD-1, PD-L1, PD-L2, ICOS, ICOSL, and CTLA-4, are administered with a time separation of more than about 60 minutes. For example, the time between the sequential administration of the additional therapeutic agent and the IAP and/or a composition comprising one or more CPIs, including but not limited to an agent that modulates one or more of PD-1, PD-L1, PD-L2, ICOS, ICOSL, and CTLA-4, can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the IAP and/or a composition comprising one or more CPIs, including but not limited to an agent that modulates one or more of PD-1, PD-L1, PD-L2, ICOS, ICOSL, and CTLA-4, being administered. Either the additional therapeutic agent or the IAP and/or a composition comprising one or more CPIs, including but not limited to an agent that modulates one or more of PD-1, PD-L1, PD-L2, ICOS, ICOSL, and CTLA-4, may be administered first.

Co-administration also does not require the additional therapeutic agent and the IAP and/or a composition comprising one or more CPIs, including but not limited to an agent that modulates one or more of PD-1, PD-L1, PD-L2, ICOS, ICOSL, and CTLA-4, to be administered to the subject by the same route of administration. Rather, each therapeutic agent can be administered by any appropriate route, for example, parenterally or non-parenterally.

In embodiments, the additional therapeutic agent is a corticosteroid. In embodiments, the additional therapeutic is an agent that targets tumor necrosis factor alpha (TNF-α).

In embodiments, the additional therapeutic agent is an anti-bacterial agent, which includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In embodiments, the anti-bacterial agent may be any of the penicillin, cephalosporin, monobactam, and carbapenem antibiotics.

Non-limiting examples of additional therapeutic agents include analgesics, such as nonsteroidal anti-inflammatory agents, corticosteroid anti-inflammatory agents, antipruritics/local anesthetics, opiate agonists, and salicylates; anti-infective agents, such as anthelmintics, antianaerobics, antibiotics, aminoglycoside antibiotics, antifungal antibiotics, cephalosporin antibiotics, macrolide antibiotics, miscellaneous B-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H2-blocker anti-ulcer agents, cholelitholytic agents, digestants, emetics, laxatives and stool softeners, and prokinetic agents; gastrointestinal hormones and hormone modifiers, such as GLP-1, GLP-2, abortifacients, adrenal agents, corticosteroid adrenal agents, androgens, anti-androgens; nutritional supplementation agents such as minerals and/or vitamins, such as water soluble or fat soluble vitamins, vitamin A, vitamin B, vitamin C, vitamin D, vitamin E, and/or vitamin K, and metabolizable carbohydrates, amino acids, and/or fats.

In embodiments, the one or more additional therapeutic agents includes fecal microbiota, for example from a fecal transplant to reshape the gut microbiome for therapeutic purposes. In embodiments, the one or more additional therapeutic agents includes any therapeutic approved for one or more diseases and/or disorders described herein.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents (e.g., microbiome-modulating agents and/or additional therapeutic agents described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In embodiments, compositions and methods that exhibit large therapeutic indices are preferred. A therapeutically effective dose can be estimated initially from in vitro assays, including, for example, cell culture assays or measurements or methane production in stool samples. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

Hereinafter, the present disclosure will be described in further detail with reference to examples. These examples are illustrative purposes only and are not to be construed to limit the scope of the present invention. In addition, various modifications and variations can be made without departing from the technical scope of the present invention.

### EXAMPLES

### Example 1: Treatment of Immune Checkpoint Inhibitors (CPI)-Mediated Gastrointestinal Side Effects

A clinical study of treatment of CPI-mediated gastrointestinal side effects are carried out. The study will include subjects who are undergoing therapy with an immune checkpoint inhibitor immunotherapy selected from an agent that modulates one or more of programmed cell death protein-1 (PD-1) (e.g., an antibody or antibody format specific for PD-1, e.g., Nivolumab, Pembrolizumab, or Pidilizumab), programmed death-ligand 1 (PD-L1) (e.g., an antibody or antibody format specific for PD-L1, e.g., BMS-936559, Atezolizumab, Avelumab, or Durvalumab), programmed death-ligand 2 (PD-L2) (e.g., an antibody or antibody format specific for PD-L2), inducible T-cell costimulator (ICOS) (e.g., an antibody or antibody format specific for ICOS, e.g., JTX-2011), inducible T-cell costimulator ligand (ICOSL) (e.g., an antibody or antibody format specific for ICOSL), and cytotoxic T-lymphocyte-associated protein 4 (CTLA-4) (e.g., an antibody or antibody format specific for CTLA-4, e.g., tremelimumab or Ipilimumab).

Any of the subjects may be cancer patients, wherein the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including GI cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), or Meigs' syndrome.

The subjects will be divided into one or more test groups and a control group. Both the control group and the one or more test groups will be administered an immune checkpoint inhibitor immunotherapy, and the one or more test groups will additionally be orally administered a therapeutically effective amount of an IAP having the amino acid sequence of SEQ ID NO:2 or SEQ ID NO:11, before or after administration of the immune checkpoint inhibitor immunotherapy.

The subjects will be monitored for CPI-mediated gastrointestinal side effects, e.g.:
Diarrhea and/or colitis
Anti-PD-1/anti-CTLA-4: Inflammatory infiltrate in the lamina propria composed of lymphocytes, neutrophils, eosinophils and plasma cells; neutrophilic crypt abscess formation; increased apoptotic activity within the crypt epithelium; crypt epithelial atrophy and crypt dropout. Chronic inflammatory changes including crypt distortion, basal plasmacytosis and paneth cell metaplasia. Granulomas. Lymphocytic colitis and collagenous colitis.
Anti-PD-1: Features of acute colitis; chronic colitis (basal lymphoplasmacytosis and crypt architectural irregularity, paneth cell metaplasia); crypt abscesses; apoptosis; inflammatory infiltrate in the lamina propria composed of lymphocytes, neutrophils, eosinophils and plasma cells. Lymphocytic colitis and collagenous colitis.
Anti-CTLA-4: Features of acute colitis; chronic colitis (basal lymphoplasmacytosis and crypt architectural irregularity, paneth cell metaplasia); neutrophilic inflammation only; lymphocytic inflammation only; combined neutrophilic and lymphocytic infiltration; intra-epithelial neutrophilic lymphocytes; cryptitis; crypt abscesses; apoptosis; inflammatory infiltrate in the lamina propria composed of lymphocytes, neutrophils, eosinophils and plasma cells; granulomas. Lymphocytic colitis.

The test group is expected to exhibit improvements over the control group, e.g., one or more of the following:
Reduction or prevention of one of the CPI-mediated gastrointestinal side effects listed above.
Prevention or reduction of diarrhea and/or colitis.
Reduction in dose or frequency of corticosteroid administration needed for mediated GI side effect treatment.
Obviation of corticosteroid administration for GI side effect treatment.
Reduction in dose or frequency of administration of one or more agents targeting TNF-α needed for mediated GI side effect treatment.
Obviation of administration of one or more agents targeting TNF-α for GI side effect treatment.
Increase in the therapeutic window of the CPI.

### EQUIVALENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

1. A method for preventing or reducing an immune checkpoint inhibitors (CPI)-mediated gastrointestinal side effect in a patient in need thereof, comprising administering to the patient an intestinal alkaline phosphatase (IAP),
wherein the patient is undergoing therapy with an immune checkpoint inhibitor immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

2. A method for preventing an immune checkpoint inhibitors (CPI)-mediated gastrointestinal (GI) side effect in a patient in need thereof, comprising administering to the patient an intestinal alkaline phosphatase (IAP) and a CPI selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

3. The method of claim 2, wherein the IAP is administered before the CPI.

4. The method of any of the above claims, wherein the CPI-mediated GI side effect is diarrhea and/or colitis.

5. The method of any of the above claims, wherein the IAP is selected from human IAP or calf/bovine IAP.

6. The method of any of the above claims, wherein the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with any one of SEQ ID NOs: 1-6, or 10-14.

7. The method of any of the above claims, wherein the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with SEQ ID NO: 2.

8. The method of any of the above claims, wherein the IAP comprises an amino acid sequence having at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99%, or at least about 100% identity with SEQ ID NO: 11.

9. The method of any of the above claims, wherein the IAP is administered orally.

10. The method of any of the above claims, wherein the IAP is formulated for GI release.

11. The method of any of the above claims, wherein the agent that modulates PD-1 is an antibody or antibody format specific for PD-1.

12. The method of claim 11, wherein the antibody or antibody format specific for PD-1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

13. The method of claim 11, wherein the antibody or antibody format specific for PD-1 is selected from Nivolumab, Pembrolizumab, and Pidilizumab.

14. The method of any of the above claims, wherein the agent that modulates PD-L1 is an antibody or antibody format specific for PD-L1.

15. The method of claim 14, wherein the antibody or antibody format specific for PD-L1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

16. The method of claim 14, wherein the antibody or antibody format specific for PD-L1 is selected from BMS-936559, Atezolizumab, Avelumab and Durvalumab.

17. The method of any of the above claims, wherein the agent that modulates PD-L2 is an antibody or antibody format specific for PD-L2.

18. The method of claim 17, wherein the antibody or antibody format specific for PD-L2 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

19. The method of any of the above claims, wherein the agent that modulates ICOS is an antibody or antibody format specific for ICOS.

20. The method of claim 19, wherein the antibody or antibody format specific for ICOS is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

21. The method of claim 19, wherein the antibody or antibody format specific for ICOS comprises JTX-2011.

22. The method of any of the above claims, wherein the agent that modulates ICOSL is an antibody or antibody format specific for ICOSL.

23. The method of claim 22, wherein the antibody or antibody format specific for ICOSL is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

24. The method of any of the above claims, wherein the agent that modulates CTLA-4 is an antibody or antibody format specific for CTLA-4.

25. The method of claim 24, wherein the antibody or antibody format specific for CTLA-4 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

26. The method of claim 24, wherein the antibody or antibody format specific for CTLA-4 is selected from tremelimumab and Ipilimumab.

27. The method of any of the above claims, wherein the patient is a cancer patient.

28. The method of any of the above claims, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including GI cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

29. The method of any of the above claims, further comprising administering one or more corticosteroids.

30. The method of any of the above claims, wherein the IAP treatment reduces the dose or frequency of corticosteroid administration needed for mediated GI side effect treatment.

31. The method of any of the above claims, wherein the IAP treatment obviates corticosteroid administration for GI side effect treatment.

32. The method of any of the above claims, further comprising administering one or more agents targeting tumor necrosis factor alpha (TNF-α).

33. The method of any of the above claims, wherein the IAP treatment reduces the dose or frequency of administration of one or more agents targeting TNF-α needed for mediated GI side effect treatment.

34. The method of any of the above claims, wherein the IAP treatment obviates administration of one or more agents targeting TNF-α for GI side effect treatment.

35. The method of any one of claims 31-33, wherein the agent targeting TNF-α is an antibody or fusion protein.

36. The method of claim 34, wherein the agent targeting TNF-α is infliximab (Remicade), infliximab-dyyb (Inflectra), infliximab-abda (Renflexis) or Flixabi.

37. The method of any of the above claims, wherein the method increases a therapeutic window of the CPI.

38. The method of any of claims 27-37, wherein the IAP does not hinder cancer treatment in the patient.
